(19) United States
(12) Patent Application Publication
(10) Pub. No.: US 2009/0069418 A1
(43) Pub. Date: Mar. 12, 2009

(54) Compositions and Methods for the Treatment of Disorders Associated with Aberrant Vasodilation

(76) Inventors: Nabil J. Alkayed, West Linn, OR (US); Jeffrey J. Iliff, Portland, OR (US); Wenri Zhang, Portland, OR (US); Michael Hutchens, Portland, OR (US); Matthias J. Merkel, Portland, OR (US); Donna M. Van Winkle, Portland, OR (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/109,722

(22) Filed: Apr. 25, 2008

Related U.S. Application Data
(60) Provisional application No. 60/913,925, filed on Apr. 25, 2007.
(60) Provisional application No. 60/948,225, filed on Jul. 6, 2007.

(51) Int. Cl. A61K 31/336; A61P 25/06

(52) U.S. Cl. 514/475

(57) ABSTRACT

Methods and compositions for the treatment of conditions associated with improper vasodilation are provided.

This application claims priority under 35 U.S.C. §119(e) to U.S. Provisional Patent Application No. 60/913,925, filed on Apr. 25, 2007, and U.S. Provisional Patent Application No. 60/948,225, filed on Jul. 6, 2007. The foregoing applications are incorporated by reference herein.

Pursuant to 35 U.S.C. Section 202(c), it is acknowledged that the United States Government has certain rights in the invention described herein, which was made in part with funds from the National Institutes of Health, National Institute of Neurological Disorders and Stroke Grant No. 2R01NS044313-07 and the National Institutes of Health Grant No. R01 NS44313.

### FIELD OF THE INVENTION

The present invention relates to the treatment and prevention of disorders associated with vasodilation. More specifically, the invention relates to the treatment and/or prevention of conditions characterized by ischemia-reperfusion, conditions characterized by cerebral hyperperfusion such as migraines, conditions characterized by cerebral hypoperfusion such as stroke, and conditions characterized by inappropriate vasodilation such as in the post-cardiac arrest state, through the modulation of the epoxyeicosatrienoic acid (EETs) signaling pathway.

### BACKGROUND OF THE INVENTION

Several publications and patent documents are cited throughout the specification in order to describe the state of the art to which this invention pertains. Each of these citations is incorporated by reference herein as though set forth in full.

Cerebral blood flow (CBF) is intricately regulated through multiple mechanisms that operate at different hierarchical levels to maintain adequate tissue perfusion and prevent wide fluctuations in brain blood flow. Locally, cerebral blood vessels respond to changes in their physical (intraluminal pressure, longitudinal shear) and chemical (pH, PO₂, PCO₂) environments, in addition to sensing and responding to changes in neuronal activity in order to fine-tune and dynamically regulate blood flow rates in accordance with local metabolic demands. This mechanism of blood flow regulation occurs within the so called “neurovascular unit” comprised of neurons, vascular smooth muscle (VSM), endothelium, and intervening astrocytes. These different cell types work together to match blood flow with neuronal demands, a process termed “neurovascular coupling” which forms the basis of functional magnetic resonance imaging (fMRI) (Edvinsson et al. (1993) (1993) Cerebral bloodflow and metabolism. Raven Press, New York). Neurovascular coupling is mediated in large part by astrocytes, whose processes ensheathe both neuronal synapses and parenchymal arterioles which are linked by gap junctions into an electro-chemical syncitium (Koehler et al. (2006) J. Appl. Physiol., 100:307-317). According to this model, astrocytes sense neuronal activity through stimulation of metabotropic glutamate receptors (mGluRs), which leads to the release of vasoactive compounds such as K⁺(Filosa et al. (2006) Nat. Neurosci., 9:1397-1403) or cyclooxygenase (COX) and P450 eicosanoids (Alkayed et al. (1997) Stroke 28:1066-1072; Niwa et al. (2000) J. Neurosci., 20:763-770; Peng et al. (2002) Am. J. Physiol. Heart Circ. Physiol., 283:H2029-2037) to dilate adjacent arterioles and increase nutritive blood flow. In addition to this well-appreciated mode of flow-metabolism coupling, large conduit arteries such as the middle cerebral artery (MCA) are subject to neurogenic regulation by extrinsic perivascular nerves.

Three broad classes of nerve fibers innervate cerebral surface arteries: parasympathetic nitrergic vasodilator fibers, sympathetic adrenergic vasoconstrictor nerves, and sensory vasodilator fibers (Hamel (2006) J. Appl. Physiol., 100:1059-1064). The extrinsic perivascular nerves are believed to safeguard the brain against extreme fluctuations in CBF, such as during transient hypoperfusion or chronic hypertension. Impaired neurogenic control may also have pathophysiological consequences. For example, the recruitment of sensory and parasympathetic vasodilator fibers is believed to underlie the cortical hyperemia associated with migraine (Edvinsson et al. (2005) Brain Res., 48:438-456).

In the systemic circulation, EETs are primarily produced by vascular endothelium, where they serve as an endothelium-derived hyperpolarizing factor (EDHF; Campbell et al. (1996) Circ. Res., 78:415-423). As such, EETs play an important role in regulating tissue perfusion in several organs, including the heart, brain and kidney. However, EETs are short-lived, mainly due to metabolic conversion by soluble epoxide hydrolase (sEH) into dihydroxyeicosatrienoic acids (DHETs; Zeldin, D. C. (2001) J. Biol. Chem., 276:36059-36062). Recent reports suggest that sEH inhibition is protective against cardiovascular disease, including hypertension-related end-organ damage (Zhao et al. (2004) J. Am. Soc. Nephrol., 15:1244-1253; Dorrance et al. (2005) J. Cardiovasc. Pharmacol., 46:842-848). Furthermore, sEH gene deletion in sEH knockout (sEHKO) mice renders these mice resistant to angiotensin II-induced hypertension (Sinal et al. (2000) J. Biol. Chem., 275:40504-40510). More recently, it has been demonstrated with an isolated perfused heart that sEHKO mice exhibit improved ventricular function after myocardial ischemia (Seubert et al. (2006) Circ. Res., 99:442-450). However, the impact of sEH gene deletion on cardiac function, survival, and end-organ tissue damage in an in vivo model of whole-body ischemia had not been previously studied.

### SUMMARY OF THE INVENTION

In accordance with one aspect of the instant invention, methods for treating or preventing a conditions characterized by cerebral hyperperfusion are provided. The methods comprise administering at least one agent which inhibits the EETs signaling pathway. Conditions characterized by cerebral hyperperfusion include, without limitation, migraine, cluster headaches, and primary headaches. Agents which inhibit the EETs signaling pathway include, without limitation, agents which inhibit EETs-synthesizing enzymes, agents which inhibit the liberation of EETs from the phospholipid pool, agents which increase the activity of EETs-metabolizing proteins, and agents which inhibit the action of neurogenic EETs upon the cerebral artery. The methods may further comprise administering at least one other migraine therapeutic agent.

In another embodiment of the instant invention, methods for treating or preventing a conditions characterized by cerebral hypoperfusion are provided. The methods comprise administering an agent which activates the EETs signaling pathway. Condition characterized by cerebral hypoperfusion include, without limitation, stroke, vasospasm after subarachnoid hemorrhage, and traumatic brain injury. Agents which activate the EETs signaling pathway include, without limitation, agents which increase the activity of EETs-synthesizing enzymes, agents which increase the liberation of EETs from the phospholipid pool, agents which inhibit EETs-metabolizing proteins, and agents which increase the action of neurogenic EETs upon the cerebral artery.

In yet another aspect of the instant invention, methods for treating or preventing a conditions characterized by inappropriate vasodilation are provided. The methods comprise administering at least one agent which inhibits the EETs signaling pathway. Conditions characterized by inappropriate vasodilation include, without limitation, vasodilatory shock, the post-cardiac arrest state, and hypotension. Agents which inhibit the EETs signaling pathway include, without limitation, agents which inhibit EETs-synthesizing enzymes, agents which inhibit the liberation of EETs from the phospholipid pool, and agents which increase the activity of EETs-metabolizing proteins.

In still another embodiment, methods for treating or preventing (e.g., inhibiting) ischemia-reperfusion injury are provided. The methods comprise administering an agent which activates the EETs signaling pathway. Agents which activate the EETs signaling pathway include, without limitation, agents which increase the activity of EETs-synthesizing enzymes, agents which increase the liberation of EETs from the phospholipid pool, and agents which inhibit EETs-metabolizing proteins. In particular, the methods comprise the depletion of the activity of sEH. Conditions associated with ischemia-reperfusion injury include, without limitation, surgery, transplantation (e.g., heart transplants), and coronary artery reperfusion.

In yet another embodiment, compositions for performing the above methods are provided.

### BRIEF DESCRIPTION OF THE DRAWING

FIG. 1 is a schematic drawing of the synthesis and metabolism of EETs. Epoxidation of arachidonic acid at 5-6, 8-9, 11-12 or 14-15 double bonds by cytochrome P450 (CYP450) epoxygenase enzymes results in the formation of four respective epoxyeicosatrienoic acid (EETs) regioisomers. Hydration of the epoxide by soluble epoxide hydrolase (sEH) converts EETs to dihydroxyeicosatrieonic acids (DHETs) (Zeldin, D. C. (2001) J. Biol. Chem., 276:36059-62). The schematic diagram depicts the formation and metabolism of 14,15-EET, the preferred substrate for sEH.

FIGS. 2A-2H are images demonstrating the CYP2C11 and sEH expression within the cerebral vasculature. FIG. 2A is an image showing that CYP2C11-IR is located in GFAP-positive cortical astrocytes. Utilizing confocal microscopy (3000×), CYP2C11-IR (green) is observed in the cell bodies corresponding to GFAP-positive (red) processes. FIG. 2B is an image showing sEH-IR localization in cerebral parenchymal blood vessels. Confocal microscopy (3000×), detected sEH-IR (green) within the same cell layer as the vascular smooth muscle marker myosin heavy chain I (MHC-I, red). FIG. 2C is an image of CYP2C11-IR (green) observed in cerebral perivascular nerves innervating the MCA. 400× conventional fluorescence microscopy is utilized to accentuate whole-vessel morphology. FIGS. 2D and 2E are immunofluorescent double-labeling images of CYP2C11 (green) and nNOS (red) within perivascular fibers (confocal microscopy, 600× (FIG. 2D, 3000×FIG. 2E). FIG. 2F is an image of sEH-IR observed in cerebral perivascular nerves around the MCA (400× conventional fluorescence microscopy). FIGS. 2G and 2H are immunofluorescent double-labeling images of sEH (green) and nNOS (red) within perivascular fibers (confocal microscopy, 600× (FIG. 2G), 3000× (FIG. 2H)).

FIG. 3 is a schematic drawing of two modes of CBF regulation by EETs. Within the neurovascular unit (bottom), astrocytes respond to neuronal release of glutamate (Glut) with EETs production by CYP2C11 (P450) and their subsequent release from astrocytic endfeet encompassing parenchymal arterioles. The vasodilator effect of astrocyte-derived EETs is terminated by soluble epoxide hydrolase (sEH) localized in the arteriolar vascular smooth muscle. At the level of the cerebral conduit arteries (upper), EETs may be released from parasympathetic fibers or sensory fibers originating in the otic/sphenopalatine ganglia (SPG) or trigeminal ganglia (TG), respectively. These fibers are also known to release nitric oxide (NO, parasympathetic) and calcitonin gene-related peptide (CGRP, sensory) to mediate their vasodilator effects. EETs signaling is regulated and terminated by sEH localized to the nerve fibers and the vascular smooth muscle.

FIG. 4 provides images of Western blot analyses of brain vascular and non-vascular compartments showing that sEH is predominantly expressed in brain's parenchyma, and to a lesser degree in cerebral vessels. Vascular smooth muscle α-actin is restricted to the vessel fraction, suggesting effective separation of both compartments. Western blot is representative of three blots.

FIGS. 5A-5D provide images of the localization of soluble epoxide hydrolase (sEH) immunoreactivity in mouse brain neuronal cell bodies and processes. Immunoreactivity for sEH in the cerebral cortex (FIGS. 5A to 5C) and striatum (FIG. 5D) is localized to neuronal cell bodies (dotted arrows) and processes (solid arrows). Three immunohistochemistry runs were performed on n=3 mice.

FIGS. 6A and 6B are graphs of the pharmacokinetic profile of the sEH inhibitor AUDA-BE and its metabolites in plasma. AUDA-BE was administered to mice as a single intraperitoneal injection of either 40 mg/kg (FIG. 6A) or 10 mg/kg (FIG. 6B). 12-(3-Adamantan-1-yl-ureido)-dodecanoic acid butyl ester and the metabolites AUDA and AUBA were analyzed at 1, 3, 6, and 24 hours after injection. Control injections of sesame oil without AUDA-BE led to concentrations of parent drug or metabolites below the detection limits (0.96, 1.10, and 5.78 nmol/L for AUDA, AUDA-BE, and AUBA, respectively). Control values were subtracted from corresponding values at each time point. AUBA is a biologically inactive indicator metabolite. The mean value at each time point represents two animals.

FIG. 7 is a graph demonstrating that 12-(3-Adamantan-1-yl-ureido)-dodecanoic acid butyl ester inhibits soluble epoxide hydrolase enzymatic activity in mouse brain tissue after systemic administration. Activity was measured at 1, 3, 6, and 24 hours after single AUDA-BE administration (10 mg/kg intraperitoneally) using sEH-specific surrogate substrate [³H]-trans-1,3-diphenylpropene oxide (tDPPO). Average activity over 24 hours was reduced from 1.92±0.06 nmol/mg in vehicle-treated mice to 1.56±0.13 nmol/mg protein in AUDA-BE-treated mice (n=2) at each of four time points: 1, 3, 6, and 24 hours after injection, P<0.05.

FIG. 8A is a graph showing that 12-(3-Adamantan-1-yl-ureido)-dodecanoic acid butyl ester decreases infarct size after middle cerebral artery occlusion (MCAO) in mice. Infarct size was reduced from 33±2% (n=5) in vehicle-treated mice to 20±5% (n=10) and 16±6% (n=5) when AUDA-BE was administered (10 mg/kg intraperitoneally) 1 hour before (pre) or at the time of reperfusion (post) after 2-hour MCAO. *Different from vehicle (P<0.05). FIG. 8B provides the myocardial infarct size as a percent of area at risk (I/AAR).

FIG. 9 is a graph of the P450 epoxygenase inhibitor N-methylsulfonyl-6-(2-propargylloxyphenyl) hexanamide (MS-PPOH) eliminating protection by AUDA-BE. Infract size was reduced by AUDA-BE alone (10 mg/kg, a single intraperitoneal injection at reperfusion) from 33±2% to 16±6% (n=5 per group). However, when combined with MS-PPOH (0.5 mg/200 mL over 24 hours before MCAO, via subcutaneously implanted osmotic minipumps), AUDA-BE loses it protective effect (infarct size 34±7%, n=5, not different from vehicle). *Different from vehicle (P<0.05).

FIGS. 10A and 10B show the blood flow rates distribution in mouse brain during MCAO. Flow rates were quantified by [¹⁴C]-iodoantipyrine (IAP) autoradiography at the end of 2-hour MCAO in mice treated with vehicle (sesame oil, n=5) or AUDA-BE (10 mg/kg intraperitoneally, 30 minutes before MCAO, n=5). FIG. 10A is a grey scale distribution of regional CBF rates. FIG. 10B represents the blood flow distribution in the ipsilateral hemisphere. No differences were observed in the amount of tissue (mm³) perfused with any given flow rate (mL/100 g per minute) between vehicle- and AUDABE-treated mice (n=5 per group).

FIG. 11A is a graph of the total epinephrine dose required for resuscitation. Epinephrine was delivered in divided doses while chest compressions were underway. sEHKO mice required significantly more epinephrine than WT mice (26.0±4.9 mcg, n=12, as compared to 12.0±0.5 mcg, n=15, respectively, mean±SEM). *p=0.003. FIG. 11B is a graph of the time to restoration of spontaneous circulation (ROSC). The time was measured from initiation of cardiopulmonary resuscitation (CPR) to ROSC. Chest compressions were delivered at a rate of 300/minute at the same pressure by the same investigator. Surviving sEHKO mice required longer CPR time than WT mice (163.3±28.9 (n=12) compared to 110.3±8.2 (n=15) seconds, respectively, p=0.06).

FIG. 12A is a graph of the percent survival at 10 minutes and 24 hours after cardiac arrest (CA) and CPR. Each bar represents 15 animals. Bar portion above the X axis represents survival, portion below represents mortality. sEHKO mortality was 44% and 100% at 10 minutes and 24 hours compared to 7% and 20% in WT, respectively. *p=0.014, t p<0.0001. FIG. 12B is a graph of the mean arterial blood pressure (MAP) in the minute period after CA and CPR. sEHKO mice recover from cardiac arrest with lower 1-minute MAP (86±7 mmHg vs. 103±11 mmHg in WT), achieve lower and more delayed peak (107±3 mmHg at 5 minutes vs 112±3 mmHg at 3 minutes in WT) and have lower MAP at 10 minutes post ROSC (62±8 mmHg vs. 75±9 mmHg, n=3 per group, mean±SEM).

FIG. 13A is a graph of sEH activity measured via 14,15-DHET production. Shown is the relative production of 14,15-DHET for human wild-type sEH (hrsEHWT) and two SNPs compared to naïve murine cardiomyocytes after spiking with 1 μM 14,15-EET (mean±SEM; n=3). FIG. 13B is a graph of the EPHX2 polymorphisms affects ischemic tolerance against oxygen-glucose deprivation (OGD) in isolated cardiomyocytes in vitro. Shown is the percentage of dead cells after 3 hours of reoxygenation corrected to oxygenated control (n=5 replicas, mean±SEM).

FIGS. 14A and 14B provide graphs which demonstrate ischemic tolerance of cardiomyocytes against OGD increases after inhibition of sEH activity or 14,15-EET over supply. Shown is the percentage of dead cells, corrected to oxygenated control for hsEH, wherein excess 14,15-EET (1 μM), the substrate of sEH, (FIG. 14A) or 4-phenylchalcone oxide (4-PCO), sEH inhibitor, (FIG. 14B) was added (n=4 replica, mean±SEM). FIG. 14C is a graph showing the percentage of dead cells after OGD and administration of 14,15-EET, the 14,15-EET analogue, 14,15-epoxyeicosa-5(Z)-enoic acid (14,15-EEZE), AUDA, and/or 4-PCO.

FIG. 15 is a graph depicting that sEH depletion reduces infarct size after regional myocardial I/R. Shown is the infarct size (I) as a percentage of area at risk (AAR) for wild-type C57BL\6J (WT), sEH knockout (sEHKO) and WT animals pretreated with δ-opioid-agonist, SNC-162 (WT_{SNC}). I/AAR is significantly reduced in sEHKO mice. The reduction is comparable to opioid-induced cardioprotection (mean±SEM, n=5-10 animals per group, p<0.05).

FIGS. 16A-16D are graphs which demonstrate that 14,15-EET and sEH inhibition reduce infarct size after myocardial I/R. Shown is the infarct size (I) as a percentage of area at risk (AAR) for the following treatment conditions tested: 14,15-EET injection (IV) prior to ischemia (FIG. 15A); 14,15-EET injection (IV) prior to reperfusion (FIG. 15B); AUDA-BE injection (IP) prior to ischemia (FIG. 15C); and AUDA-BE injection (IP) prior to reperfusion (FIG. 15D) compared to the corresponding vehicle (mean±SEM, n=4-8 animals per group, *p<0.05 compared to vehicle). Vehicle for EET: ethanol 25% and vehicle for AUDA-BE: sesame oil.

FIG. 17A provides images of cells demonstrating that sEH is localized in cardiomyocytes from left ventricular tissue. Shown is the immunoreactivity for sEH (first column), alpha smooth muscle actin (Alpha-SMA) for smooth muscle cells and fibromyoplasts (second column), DAPI for nuclear staining (third column) and the merged image (fourth column) for left ventricular tissue from wild-type C57BL\6J (WT—first row), sEH knockout (sEHKO—second row) and for WT without primary sEH antibody (third row). FIG. 17B is an image of an immunoblot for sEH protein expression in left ventricular sample from wild-type (WT) and sEH knockout (sEHKO) from male and female animals (n=2).

FIG. 18A is a graph of the LC-MS/MS analysis of 14,15-EET in brain extracts from WT and sEHKO mice. The upper panel extracted ion chromatograms (XICs) monitor for 1000 pg 14,15-EET-d₈ spiked into brain extracts, whereas the lower panel XICs monitor for endogenous 14,15-EET peak in sEHKO versus WT mouse brain extracts. Detection of 14,15-EET was carried out with LC-MS/MS monitoring for the transitions from m/z 319 parent ion to m/z 175 and 219 product ions. The first peak detected at 27.3 minutes with both transitions was 14,15-EET; separation of the 14,15-EET peak was ensured from a closely eluting unknown molecule peak that was also detected. Shown are the extracted ion chromatograms monitoring for m/z 319>175 from representative sEHKO (−/−) and WT (+/+) brains. FIG. 18B is an image demonstrating that sEHKO mice sustain smaller brain infarcts after MCAO compared to C57 Black WT mice. FIG. 18B is a a representative TTC-stained brain slices from WT (left) and sEHKO (right) mouse brains. FIG. 18C is a graph showing that the infarct size was reduced from 36±3% in WT mice to 16±6% (n=5 per group, manSEM) in sEHKO mice. *Different than vehicle (P<0.05).

FIG. 19A provides a graph showing that the administration of 14,15-EET reduces infarct size after MCAO compared to vehicle-treated mice (n=4 per group, *P<0.05 versus vehicle). 14,15-EET (1 μg) was administered via osmotic minipumps over 24 hours. The pump was implanted subcutaneously 1 hour before MCAO and connected to the jugular vein via catheter, and 14,15-EET was diluted in 10% ethanol in saline. FIG. 19B is a graph of the Laser-Doppler cerebrocortical tissue perfusion monitored during MCAO in WT and sEHKO mice (n=5 per group). Laser-Doppler perfusion was decreased in both groups to a similar degree on occlusion. However, while perfusion remained stable during MCAO in WT mice, in sEHKO mice, laser-Doppler slowly recovered and was significantly higher than WT starting at 30 minutes after MCAO onset. *Different than WT (P<0.05).

FIG. 20A is an image showing that the blood flow rates are higher in sEHKO compared to WT mouse brain during MCAO. Flow rates were quantified by [¹⁴C]iodoantipyrine (IAP) autoradiography at the end of 2-hour MCAO. The distribution of regional CBF rates in WT (left) and sEHKO (right) mice are shown. FIG. 20B is an image showing the immunohistochemical localization of sEH in cerebral blood vessels.

### DETAILED DESCRIPTION OF THE INVENTION

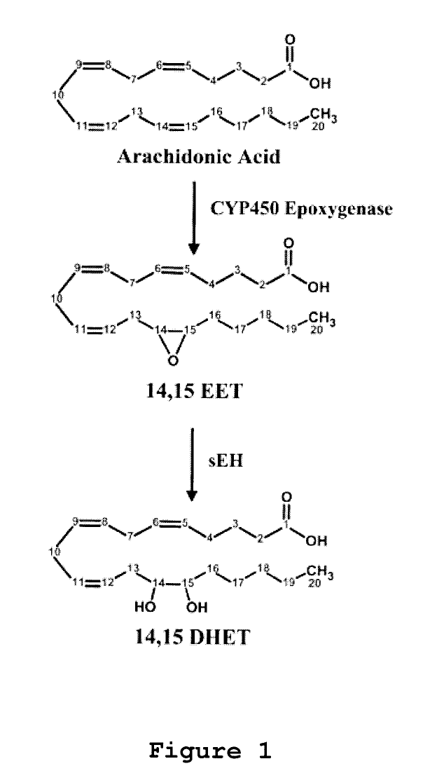

Cytochrome P450 epoxygenases catalyze the formation of EETs from arachidonic acid (AA) via the epoxidation of one of four AA double bonds, resulting in four regioisomers of EETs: 5,6-EET, 8,9-EET, 11,12-EET and 14,15-EET (FIG. 1; Roman (2002) Physiol. Rev., 82:131-185). In the brain, EETs act as vasodilators (Ellis et al. (1990) Am. J. Physiol., 259:H1171-1177) and are released from astrocytes following glutamate receptor activation (Alkayed et al. (1996) Stroke 27:971-979; Alkayed et al. (1997) Stroke 28:1066-1072). Inhibition of EETs synthesis blocks functional hyperemia in brain (Alkayed et al. (1997) Stroke 28:1066-1072; Peng et al. (2002) Am. J. Physiol. Heart Circ. Physiol., 283:H2029-2037; Peng et al. (2004) J. Cereb. Blood Flow Metab., 24:509-517), prompting the proposal that EETs are astrocyte-derived mediators of neurovascular coupling (Harder et al. (1998) Stroke 29:229-234). Though principally known for their potent vasodilator action, EETs also exert anti-inflammatory, anti-pyretic, antithrombotic and pro-angiogenic effects (Larsen et al. (2006) Eur. J. Clin. Invest., 36:293-300), in addition to conferring protection against ischemic injury (Liu et al. (2005) J. Cereb. Blood Flow Metab., 25:939-948; Gross et al., (2007) J. Mol. Cell Cardiol., 42:687-91).

The exogenous administration of EETs as therapeutic agents is generally hampered by their chemical instability and short half-life. As an alternative, the metabolic pathways of EETs can be targeted as a means of increasing the bioavailability of endogenous brain EETs. The biological effects of EETs are terminated through multiple pathways, including hydration to dihydroxyeicosatrienoic acids (DHETs) by soluble epoxide hydrolase (sEH, FIG. 1). Accordingly, the inhibition of EETs hydration by pharmacological blockade or gene deletion of sEH is a means of increasing EETs and EETs-mediated activity. For example, both sEH inhibition and gene deletion are protective against ischemic brain damage. Marked preservation of brain blood flow during the ischemic period in sEH knockout (sEHKO) mice was observed compared to wild type controls. This effect can be ascribed to an increase in the bioavailability of EETs and their vasodilator action within the cerebral circulation.

The pain that is associated with migraine headaches is the result of aberrant activation of cerebral perivascular vasodilator fibers in the linings of the brain. This activation leads to uncontrolled cerebral vasodilation and a malignant hyperemia within these blood vessels that is sensed by the brain and interpreted as intense pain. One approach in the prevention or treatment of migraine pain is to interrupt the signaling pathways between these extrinsic perivascular vasodilator fibers and the large surface cerebral conduit arteries that are their target. An example of this strategy is the tryptan class of drugs, which are calcitonin gene-related peptide (CGRP) receptor antagonists. CGRP is a potent vasodilator peptide released from the perivascular vasodilator nerves in question. Any perivascular vasodilator nerve-derived relaxing factor involved in the regulation of the surface cerebral vasculature by these fibers would be suitable for use in the methods for migraine treatment, either as a stand-alone therapy or in combination with those interacting with other parallel signaling pathways. The current invention encompasses the discovery of novel signaling molecules produced by perivascular nerves surrounding large brain surface vessels and methods of modulating the function of the same.

Epoxyeicosatrienoic acids (EETs) are vasodilators in the brain and are believed to play a role in matching cerebral blood supply with the brain's demand for nutrients and oxygen. The current view within the field of cerebral blood flow and metabolism is that EETs in the brain are produced both by an auxiliary cell type (i.e., astrocytes) which mediate communication between the brain circuitry and the blood vessels that feed them. Astrocytes also produced by the vascular endothelium (i.e., the cells lining blood vessels) which are involved in blood vessel auto-regulation. Thus, while EETs are known to be produced within the brain and play a role in cerebral blood flow regulation, that role is believed to be very local and to involve small cerebral arterioles. Further, the release of EETs as a signaling molecule from neurons has not been previously suggested or demonstrated.

As described hereinbelow, proteins responsible for EETs synthesis such as cytochrome P450 epoxygenase 2C11 (CYP2C11) and for EETs degradation such as soluble epoxide hydrolase (sEH) were determined to be located in cerebral extrinsic perivascular vasodilator nerves surrounding large arteries such as the middle cerebral artery and basilar within the linings of the brain. These nerve fibers are known to regulate the blood flow through these arteries and the presence of proteins involved in EETs synthesis (CYP2C11) and degradation (sEH) indicate that these nerves produce vasodilator EETs as a signaling molecule to mediate their effects on their target arteries.

The notion that EETs are produced by neurons runs contrary to the established dogma within the EETs/blood flow field. Cerebral blood flow is regulated in a hierarchal manner. At the lowest, most local level, vessels undergo auto-regulation intrinsically responding to changes in their local environment. At the intermediate but still local level, changes in blood flow through arteries and arterioles is coupled to changes in metabolic demand within the brain through a process termed neurovascular coupling. Overarching these two local mechanisms of cerebral blood flow regulation is the central regulation of flow through large surface arteries by perivascular nerve fibers. This system allows for an ‘override’ mechanism to force changes in blood flow in order to protect the brain against failure of the local regulatory mechanisms to maintain adequate blood flow. While EETs have been suggested in these local mechanisms involving astrocytes and the vascular endothelium, the involvement of EETs in the topmost level of cerebral blood flow regulation by central nerve-based pathways has not been previously suggested.

The perivascular vasodilator nerves (sensory fibers originating in the trigeminal ganglia and parasympathetic fibers from the otic and sphenopalatine ganglia) are known to play a key role, through their over-activation, in creating the malignant hyperemia that is the cause of pain associated with migraine headaches. Herein, it has been determined that EETs are a signaling molecule produced by these vasodilator nerves that act upon surface cerebral arteries to relax them and increase blood flow through them. The positioning of the EETs signaling system within this nerve-vessel pathway makes it a target for pharmacological intervention in the treatment of diseases associated with over-overactivation of these nerves such as migraine, cluster headaches, and other species of primary headache. Towards this end, this neurogenic EETs signaling system (as distinct from vasogenic or gliogenic) may be inhibited in the treatment of migraine-associated cerebral hyperemia. The EETs signaling pathway may be inhibited by specific inhibition of EETs-synthetic enzymes (e.g., P450 epoxygenases such as CYP2C11), by inhibition of proteins involved in liberation of EETs from the phospholipid pool (e.g., phospholipase C), activation of the EETs-metabolizing sEH, and preventing the action of neurogenic EETs upon the cerebral artery through the use of putative EETs receptor antagonists or EETs mimetic compounds. Inhibition of the EETs signaling pathway may be accomplished, for example, by the administration of chemical compounds, drugs, proteins, peptides, nucleic acid molecules encoding a protein or peptide of interest, and/or inhibitory nucleic acid molecules (e.g., siRNAs and antisense oligonucleotides).

While shutting down the EETs signaling pathway can be used to treat and/or prevent conditions characterized by cerebral hyperperfusion such as migraines, the EETs signaling pathway may also be activated to treat and/or prevent conditions characterized by hypoperfusion such as stroke, vasospasm after subarachnoid hemorrhage, and traumatic brain injury. The EETs signaling pathway may be activated/increased/stimulated by increasing the activity of EETs-synthetic enzymes (e.g., P450 epoxygenases such as CYP2C11), by increasing the activity of proteins involved in liberation of EETs from the phospholipid pool (e.g., phospholipase C), inhibiting the EETs-metabolizing sEH, and increasing the activity of neurogenic EETs upon the cerebral artery through the use of putative EETs receptor agonists. The activation of the EETs signaling pathway may be accomplished, for example, by the administration of chemical compounds, drugs, proteins, peptides, nucleic acid molecules encoding a protein or peptide of interest, and/or inhibitory nucleic acid molecules (e.g., siRNAs and antisense oligonucleotides).

Currently, other vasodilators such as nitric oxide and calcitonin gene-related peptide are known to be released from these cerebral perivascular vasodilator nerve fibers in the control of cerebral blood flow. One of these, CGRP, is the basis of a clinically used treatment for migraine, the tryptan class of drugs. The targeting of the EETs signaling pathway may be used either in parallel with anti-CGRP treatment (e.g., for non-responders) or in combination with this treatment regime.

One substantial benefit to targeting neurogenic EETs synthesis in the treatment or prevention of the above identified disorders is the ability to modulate cerebral blood flow regulation at numerous levels of the cerebrovascular tree. EETs are known to be produced by blood vessels themselves and play a role in the autoregulatory processes. They are also produced by astrocytes and are involved in the process of neurovascular coupling (the local control of blood flow). By targeting EETs at the top hierarchal level of blood flow regulation (the central control by perivascular nerves), the benefit from the modulation of EETs signaling at lower hierarchal levels would also be expected. Such a strategy for intervention involving the top-down recruitment of a single signaling pathway in the treatment of migraine is absent from currently available treatments.

As described hereinbelow, an in vivo mouse model of cardiac arrest (CA) followed by cardiopulmonary resuscitation (CPR) to determine if sEHKO mice demonstrate improved survival and functional recovery and attenuated peripheral organ damage. In contrast to previous findings described hereinabove, sEH gene deletion, which protects against ischemic damage in isolated heart preparation, impedes CPR and worsens survival after CA in vivo.

Accordingly, the administration of at least one agent which inhibits the EETs signaling pathway can be used to treat and or prevent conditions characterized by inappropriate vasodilation such as vasodilatory shock, the post-cardiac arrest state, and hypotension. Agents which inhibit the EETs signaling pathway include, without limitation, agents which inhibit EETs-synthesizing enzymes, agents which inhibit the liberation of EETs from the phospholipid pool, agents which increase the activity of EETs-metabolizing proteins, and agents which inhibit the action of neurogenic EETs upon arteries.

In accordance with another aspect of the instant invention, methods are provided for the treatment or inhibition of ischemia-reperfusion injury. These methods comprise increasing the level of available 14,15-EET. For example, the coronary infusion of 14,15-EET or analogs thereof and/or sEH inhibitors can inhibit ischemia-reperfusion injury, such as during cardiac surgery, heart transplantation, and coronary artery reperfusion. Further, infusion of 14,15-EET or analogs thereof and/or sEH inhibitors into the coronary system during cardiopulmonary bypass (e.g., with standard cardioplegic solution) can be used to induce cardioprotection against ischemia-reperfusion injury. 14,15-EET or analogs thereof and/or sEH inhibitors may also be administered (e.g., locally) during cardiac surgery to induce cardioprotection against ischemia-reperfusion injury. 14,15-EET or analogs thereof and/or sEH inhibitors may also be administered (e.g., to the organ/tissue and/or to the recipient) to increase organ function and decrease tissue damage during ischemia time of donated organs (e.g., heart). Coronary infusion of 14,15-EET or analogs thereof and/or sEH inhibitors may also be used during coronary artery reperfusion in patients experiencing coronary arterial occlusion (e.g., heart attack) to induce cardioprotection against ischemia-reperfusion injury. Indeed, coronary infusion of 14,15-EET or analogs thereof and/or sEH inhibitors may be administered to patients undergoing percutaneous coronary interventions (PCI) to elicit reperfusion of coronary artery occlusion to induce cardioprotection against ischemia-reperfusion injury. The administration of 14,15-EET or analogs thereof and/or sEH inhibitors will generally improve cardiac function, reduce myocardial damage, reduce mortality and morbidity, and reduce the length of inotropic support for patients undergoing cardiac surgery or transplants. As demonstrated hereinbelow, 14,15-EET and/or sEH inhibitors may be administered prior to ischemia, prior to reperfusion or soon after reperfusion. In a preferred embodiment, the 14,15-EET and/or sEH inhibitor is administered at least prior to reperfusion. In another embodiment, 14,15-EET is administered prior to ischemia.

### I. DEFINITIONS

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

A “replicon” is any genetic element, for example, a plasmid, cosmid, bacmid, plastid, phage or virus, which is capable of replication largely under its own control. A replicon may be either RNA or DNA and may be single or double stranded.

A “vector” is a replicon, such as a plasmid, cosmid, bacmid, phage or virus, to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The terms “percent similarity”, “percent identity” and “percent homology” when referring to a particular sequence are used as set forth in the University of Wisconsin GCG software program.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

The term “oligonucleotide” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

Antisense molecules are typically between about 15 and about 30 nucleotides in length and often span the translational start site of mRNA molecules. Antisense constructs may also be generated which contain the entire gene sequence in reverse orientation. Antisense oligonucleotides targeted to any known nucleotide sequence can be prepared by oligonucleotide synthesis according to standard methods.

Typically, siRNA molecules are double stranded RNA molecules between about 12 and 30 nucleotides in length, more typically about 21 nucleotides in length (see Ausubel et al., eds. Current Protocols in Molecular Biology, John Wiley and Sons, Inc., (2005)).

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA molecule of the invention, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., 1989, Molecular Cloning: A Laboratory Manual, Cold Spring Harbor Laboratory Press):

Tₘ=81.5° C.+16.6 Log [Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, or the addition of stabilizers.

The term “migraine” refers to a severe episodic headache characterized by at least one of the following symptoms: unilateral, intense pulsating headache, nausea, vomiting, sparkling, rainbow-like colors, blank spots in the field of vision, and other auras and sensitivity to light and sounds. A migraine may be diagnosed by determining whether some of a person's recurrent headaches meet migraine criteria as disclosed in The International Classification of Headache Disorders, 2nd edition, Headache Classification Committee of the International Headache Society: Cephalalgia 24, supplement 1, 2004.

As used herein, a primary headache (a pain in the head, such as in the scalp, face, forehead or neck) is a headache which is not caused by another condition. Contrarily, a secondary headache is due to a disease or medical condition, such as an illness, infection, injury, stroke or other abnormality.

As used herein, a cluster headache (also known as histamine headache, histamine cephalalgia, Raedar's syndrome, or sphenopalatine neuralgia) is a vascular headache syndrome which is characterized by a series of short-lived attacks of pain. Cluster headaches are a grouping of headaches, usually occurring on a regular basis one or more times a day usually over a period of several weeks, with each headache typically brief in duration (e.g., a few moments to 2 hours) followed by a pain-free period.

The term “vasodilatory shock” refers to conditions characterized by severe venous or arteriolar dilation typically resulting in inadequate blood flow. Causes of vasodilatory shock include, without limitation, cerebral trauma, drug or poison toxicity, anaphylaxis, liver failure, heat exposure, bacteremia, and sepsis.

“Pharmaceutically acceptable” indicates approval by a regulatory agency of the Federal government or a state government. “Pharmaceutically acceptable” agents may be listed in the U.S. Pharmacopeia or other generally recognized pharmacopeia for use in animals, and more particularly in humans.

A “carrier” refers to, for example, a diluent, adjuvant, excipient, auxiliary agent or vehicle with which an active agent of the present invention is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin.

### II. Methods and Compositions

The instant invention encompasses methods and compositions for treating and/or preventing conditions characterized by cerebral hyperperfusion and conditions associated with inappropriate blood vessel dilation by inhibiting the EETs signaling pathway, i.e., decreasing the activity of EETs. Conditions characterized by cerebral hyperperfusion include, without limitation, migraine, cluster headaches, and other species of primary headache. The EETs signaling pathway may be inhibited, for example, by one or more of the following methods.

(1) EETs-synthetic enzymes may be specifically inhibited. EETs-synthesizing enzymes include P450 epoxygenases such as CYP2C11. Examples of p450 inhibitors include, without limitation, miconazole and methylsulfonyl-6-(2-proparglyoxyphenyl)hexanamide (MS-PPOH).

(2) The proteins involved in liberation of EETs from the phospholipid pool may be inhibited. For example, phospholipases such as phospholipase C and phospholipase A2 may be inhibited.

(3) The activity of EETs-metabolizing proteins may be increased. EETs-metabolizing proteins include, without limitation, sEH. sEH activity may be increased by administering sEH to a subject or by administering nucleic acid molecules encoding sEH to the subject.

sEH is encoded by the EPHX2 gene (Newman et al. (2003) Proc. Natl. Acad. Sci., 100:1558-1563; Koerner et al. (2007) J. Neurosci.). EPXH2 consists of 19 exons and is approximately 45 kb. The coding sequence corresponds to a protein of 555 amino acid residues (Sandberg et al. (1996) Biochem. Biophys. Res. Commun., 221:333-9). Soluble epoxide hydrolase has over 90% homology between rodent and human (Arand et al. (1994) FEBS Lett., 338:251-6). In humans, sEH has been localized to chromosome 8 (Larsson et al. (1995) Hum. Genet., 95:356-8). Genetic polymorphisms have also been described in the human EPXH2 (Sandberg et al. (2000) J. Biol. Chem., 275:28873-81; Przybyla-Zawislak et al. (2003) Mol. Pharmacol., 64:482-90), which have been linked to a variety of human diseases, including hypertension (Fornage et al. (2002) Hypertension, 40:485-90), hypercholesterolemia (Sato et al. (2004) J. Hum. Genet., 49:29-34), coronary artery calcification (Fornage et al. (2004) Circulation, 109:335-9), and insulin resistance in type 2 diabetes (Ohtoshi et al. (2005) Biochem. Biophys. Res. Commun., 331:347-50).

(4) The action of neurogenic EETs upon the cerebral artery may be inhibited through the use of EETs receptor antagonists or EETs mimetic compounds. An example of an EETs receptor antagonist is the 14,15-EET analogue, 14,15-epoxyeicosa-5(Z)-enoic acid (14,15-EEZE; Gauthier et al. (2002) Circ. Res., 90:1028-1036).

In a particular embodiment, compositions of the instant invention comprise at least one of the above-identified agents and a carrier.

In addition to the above agents, at least one more migraine therapeutic agent may be administered (sequentially or concurrently) or included in the compositions of the instant invention. Migraine therapeutic agents include, without limitation, analgesics (e.g., acetaminophen, aspirin, and ibuprofen), non-steroidal anti-inflammatory agents (e.g., naproxen and naproxen sodium), or specific agents such as ergotamines or triptans (e.g., serotonin agonists, 5-hydroxy tryptamine (5-HT) agonists, sumatriptan, sumatriptan succinate).

The instant invention also encompasses methods and compositions for treating and/or preventing conditions characterized by cerebral hypoperfusion and conditions associated with ischemia-reperfusion injury by activating the EETs signaling pathway, i.e., increasing the activity of EETs (e.g., by administering EETs). Conditions characterized by cerebral hypoperfusion include, without limitation, stroke, vasospasm after subarachnoid hemorrhage, and traumatic brain injury. The EETs signaling pathway may be activated, for example, by one or more of the following methods.

(1) The activity of EETs-synthetic enzymes may be specifically increased. EETs-synthesizing enzymes include P450 epoxygenases such as CYP2C11. CYP2C11 activity may be increased by administering CYP2C11 to a subject or by administering nucleic acid molecules encoding CYP2C11 to the subject. Additionally, P450 activity may be increased through the administration cholesterol lowering agents such as clofibrate. P450 activity may also be increased by hypoxic and ischemic preconditioning (Alkayed et al. (2002) Stroke 33:1677-84; Liu et al. (2005) J. Cereb. Blood Flow Metab., 25:939-48).

(2) The activity of proteins involved in liberation of EETs from the phospholipid pool may be increased. For example, the activity of phospholipases such as phospholipase C and phospholipase A2 may be increased.

(3) EETs-metabolizing proteins may be inhibited. EETs-metabolizing proteins include, without limitation, sEH. 12-(3-adamantane-1-yl-ureido)-dodecanoic acid butyl ester (N-adamantanyl-N′-dodecanoic acid urea butyl ester; AUDA-BE; Kim et al. (2004) J. Med. Chem., 47:2110-2122) is an example of a specific sEH inhibitor. Notably, AUDA-BE is capable of crossing the blood brain barrier (BBB). Chalcone oxides, such as 4-phenylchalcone oxide (4-PCO; Morisseau et al. (1998) Arch. Biochem. Biophys., 356:214-228), are also potent inhibitors of sEH, but are often rapidly metabolized in vivo. Urea, amide and carbamate derivatives have been generated, however, which are more metabolically stable (see, e.g., Kim et al. (2004) J. Med. Chem., 47:2110-2122; Smith et al. (2005) Proc. Natl. Acad. Sci., 102:2186-2191; Schmelzer et al. (2005) Proc. Natl. Acad. Sci., 102:9772-9777; Zhao et al. (2004) J. Am. Soc. Nephrol., 15:1244-1253; Dorrance et al. (2005) J. Cardiovasc. Pharmacol., 46:842-848). sEH inhibitors are also described in Morisseau et al. (Proc. Natl. Acad. Sci. (1999) 96:8849-8854), U.S. Pat. Nos. 5,955,496, 6,150,415, and 6,531,506, and U.S Patent Application Publication Nos. 2004/0092567, 2006/0276515, and 2006/0270609.

(4) The action of neurogenic EETs upon the cerebral artery may be increased through the use of EETs receptor agonists. Examples of EETS agonists include, without limitation, 14,15-EET-phenyliodosulfonamide (14,15-EET-PISA), 14,15-EET-biotinsulfonamide (14,15-EET-BSA) and 14,15-EET-benzoyldihydrocinnamide-sulfonamide (14,15-EET-BZDC-SA) (Yang et al. (2007) J. Pharmacol. Exp. Ther.).

In a particular embodiment, compositions of the instant invention comprise at least one of the above-identified agents and a carrier.

The following examples are provided to illustrate various embodiments of the present invention. They are not intended to limit the invention in any way.

### EXAMPLE 1

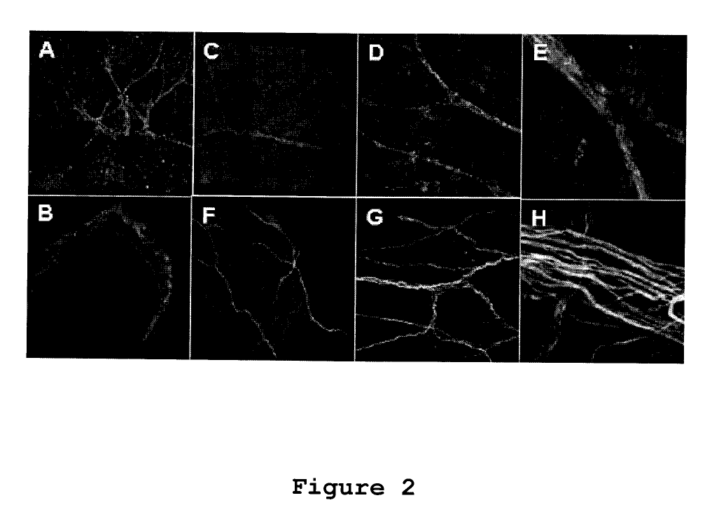

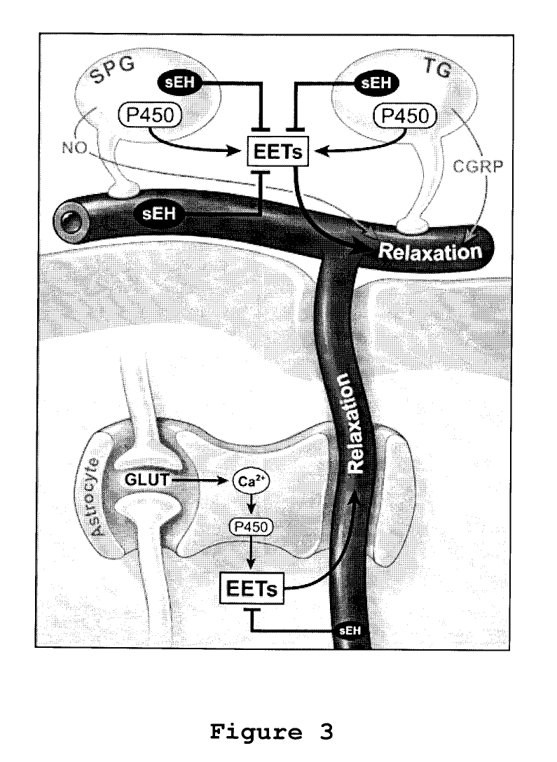

Using immunofluorescent double-labeling with cell-specific markers, sEH expression was found in cerebral parenchymal microvessels. Additionally, the presence of CYP2C11 immunoreactivity (IR) within GFAP-positive astrocytes was confirmed (FIG. 2A). In these vessels, sEH-IR co-localized with myosin heavy chain I (MHC-I), suggesting that sEH-IR is specifically expressed within vascular smooth muscle (VSM) cells (FIG. 2E). This finding is in broad agreement with results from peripheral vascular beds, where sEH expression is primarily localized within the VSM, and where it is presumed to terminate the activity of endothelium-derived vasodilator EETs (Enayetallah et al. (2006) J. Histochem. Cytochem., 54:329-335; Enayetallah et al. (2004) J. Histochem. Cytochem., 52:447-454). Accordingly, as depicted in FIG. 3, astrocytic CYP2C11 (which synthesizes EETs) and vascular smooth muscle sEH (which terminate the action of EETs) form the functional units of EETs signaling within the neurovascular unit. The increased blood flow response to vascular occlusion observed in sEHKO mice is, therefore, attributable to the loss of VSM sEH, thereby resulting in enhanced dilation by EETs within the neurovascular unit and preserved collateral blood flow during focal vascular occlusion.

The expression of sEH and CYP2C11 in whole-mount large cerebral surface vessels, such as the middle cerebral artery (MCA), was also investigated. No CYP2C11 expression was observed within the VSM or endothelium. However, sEH-IR was observed in vascular cells and exhibited the circumferential orientation characteristic of arterial VSM cells (FIG. 2B). Additionally, CYP2C11 and sEH expression was also unexpectedly observed within perivascular nerves innervating the MCA (FIGS. 2B and 2F, respectively). Innervation of the MCA by CYP2C11- and sEH-positive fibers extended along the trunks of the conduit arteries and their most proximal branches. Notably, the innervation terminated prior to distal surface branches and penetrating arterioles.

As mentioned above, three distinct populations of nerve fibers are known to innervate the cerebral vasculature at this level: parasympathetic nitrergic vasodilator fibers originating in the sphenopalatine (SPG) and otic ganglia (OG), sympathetic adrenergic vasoconstrictor fibers originating in the superior cervical ganglia (SCG), and calcitonin gene-related peptide (CGRP)-releasing sensory fibers originating in the trigeminal ganglia (TG) (Hamel (2006) J. Appl. Physiol., 100:1059-1064). Given the vasodilator properties of EETs, CYP2C11 and sEH were likely expressed within extrinsic parasympathetic and sensory vasodilator fibers innervating the MCA.

Utilizing immunofluorescent double-labeling and confocal microscopy, CYP2C11- and sEH-IR was co-localized with known markers of the parasympathetic, sympathetic and sensory nerve populations that innervate large cerebral arteries. The results are summarized in Table 1. In general, sEH-positive fibers were more numerous around the MCA than CYP2C11-positive nerves. Double labeling demonstrated that all (100%) CYP2C11-positive fibers were sEH-positive, whereas only a fraction (40%) of sEH-positive fibers co-labeled for CYP2C11 (n=3). Antibody specificity was confirmed by omitting the primary antibody, by using sEHKO mouse brain tissue, and by performing antigen competition studies.

TABLE 1

| | | | Parasympathetic | | | Sensory | | Sympathetic | |
|---|---|---|---|---|---|---|---|---|---|
| | CYP2C11 | sEH | ChAT | VIP | nNOS | CGRP | SubP | DBH | NPY |
| CYP2C11 | | ++++ | + | + | ++++ | +++ | + | − | − |
| sEH | ++ | | + | + | ++++ | +++ | + | + | + |

CYP2C11, cytochrome P450 epoxygenase isoform 2C11; sEH, soluble epoxide hydrolase; ChAT, choline acetyltransferase; VIP, vasoactive intestinal peptide; nNOS, neuronal nitric oxide synthase; CGRP, calcitonin gene-related peptide; SubP, substance P; DBH, dopamine beta-hydroxylase; NPY, neuropeptide Y.
Co-localization:
− no co-localization;
+ <25%;
++ 25-50%;
+++ 50-75%;
++++ >75%.

In addition to nitric oxide (NO), parasympathetic fibers innervating the cerebral vessels release vasoactive intestinal peptide (VIP) and acetylcholine (ACh) (Hamel (2006) J. Appl. Physiol., 100:1059-1064). Therefore, CYP2C11-IR and sEH-IR were colocalized with these parasympathetic markers. Within perivascular fibers innervating the MCA, 100% of CYP2C11-positive fibers were found to be co-labeled for nNOS (n=5, FIG. 2C-D). Fibers expressing VIP or choline acetyltransferase (ChAT) were less numerous than nNOS-positive fibers, and represented a fraction (15%, n=3 each) of CYP2C11-expressing fibers. Similarly, most sEH-positive fibers (75%, n=6) co-expressed nNOS (FIG. 2E-F), whereas ChAT and VIP-positive fibers represented a small portion (5-10%, n=3 each) of the larger sEH-positive fiber pool.

Perivascular sensory fibers also express nNOS, in addition to such peptidergic vasodilators as CGRP and substance P (SubP) (Hamel (2006) J. Appl. Physiol., 100:1059-1064). Both CYP2C11- and sEH-IR were observed to co-localize with CGRP at a high frequency (60% and 50% of fibers, respectively; n=3 each). Markedly fewer SubP- than CGW-positive fibers were observed innervating the MCA, and these fibers represented a correspondingly small proportion of CYP2C11- and sEH-expressing fibers (20% and 15%, respectively; n=2 each).

Sympathetic innervation of the cerebral conduit vessels is primarily adrenergic, although many fibers also express neuropeptide Y (NPY) (Hamel (2006) J. Appl. Physiol., 100:1059-1064). Double labeling studies demonstrated that CYP2C11 did not co-localize with the adrenergic marker dopamine β-hydroxylase (DBH) or NPY in these perivascular nerves. Similarly, sEH expression was observed only in a small portion of DBH- or NPY-positive fibers. However this coexpression was infrequent and was observed only in those fibers exhibiting the weakest sEH-IR.

In summary, the results of these studies suggest that components of the EETs signaling system, EETs-synthesizing CYP2C11 and EETs-metabolizing sEH, are present within both parasympathetic and sensory vasodilator fibers innervating the MCA. Co-expression was observed to be greatest in nNOS-positive fibers, likely comprised of nitrergic parasympathetic and sensory nerve populations.

### EXAMPLE 2

### Materials and Methods

The study was conducted in accordance with the National Institutes of Health guidelines for the care and use of animals in research, and protocols were approved by the Animal Care and Use Committee of Oregon Health and Science University (Portland, Oreg., USA).

### Middle Cerebral Artery Occlusion in Mice

Transient focal cerebral ischemia was induced in adult male C57Bl/6 mice (20 to 26 g, Charles River, Hollister, Calif., USA) using the intraluminal MCAO technique, as described previously (Alkayed et al. (2001) J. Neurosci., 21:7543-50). Briefly, mice were anesthetized with halothane (1.5 to 2% in O₂— enriched air by face mask), and kept warm with water pads. A small laser-Doppler probe was affixed to the skull to monitor cortical perfusion and verify vascular occlusion and reperfusion. A silicone-coated 6-0 nylon monofilament was inserted into the right internal carotid artery via the external carotid artery until laser-Doppler signal dropped to less than 20% of baseline. After securing the filament in place, the surgical site was closed, and the animal was awakened and assessed at 2 hours of occlusion for neurological deficit using a simple neurological scoring system as follows: 0=no deficit, 1=failure to extend forelimb, 2=circling, 3=unilateral weakness, 4=no spontaneous motor activity. Mice with clear neurological deficit were re-anesthetized, laser-Doppler probe re-positioned over same site on the skull, and the occluding filament was withdrawn to allow for reperfusion. Mice were then allowed to recover and observed for 1 day. Infarct size was measured at 24 hours after MCAO in 2-mm thick coronal brain sections (five total) using 2,3,5-triphenyltetrazolium chloride staining and digital image analysis. Sections were incubated in 1.2% 2,3,5-triphenyltetrazolium chloride in saline for 15 minutes at 37° C., and then fixed in formalin for 24 hours. Slices were photographed, and infarcted (unstained) and uninfarcted (red color) areas were measured with MCID software (InterFocus Imaging Ltd, Linton, UK) and integrated across all five slices. To account for the effect of edema, the infarcted area was estimated indirectly by subtracting the uninfarcted area in the ipsilateral hemisphere from the contralateral hemisphere, and expressing infarct volume as a percentage of contralateral hemisphere. To determine the effect of sEH inhibition on infarct size after MCAO, selective sEH inhibitor AUDA-BE (Schmelzer et al. (2005) Proc. Natl. Acad. Sci., 102:9772-7) was administered intraperitoneally 30 minutes before MCAO or right after reperfusion (10 mg/kg in 100 mL sesame oil, n=5 per group). To determine if the effect of AUDA-BE is mediated through EETs, AUDA-BE was co-administered with the P450 epoxygenase inhibitor N-methylsulfonyl-6-(2-propargyloxyphenyl)hexanamide (MS-PPOH, 0.5 mg/200 mL over 24 hours via osmotic minipump, n=5, Brand-Schieber et al. (2000) J. Physiol. Pharmacol., 51:655-72). The osmotic pumps (model 2001D; DURECT Corporation, Cupertino, Calif., USA) were implanted subcutaneously 24 hours before MCAO, and AUDA-BE was administered at the time of reperfusion. The pumps have 8.07 1.0 mL/h (s.d.) mean pumping rate, which delivers 200 mL mean fill volume over 24-hour period. Animals were randomly assigned to treatment groups.

### Immunohistochemistry

To localize sEH immunoreactivity in brain, thin coronal sections (6 mm thick) were deparaffinized and heated in 0.5 mmol/L sodium citrate buffer at pH 6.0 in a steamer for 30 minutes to intensify staining. Sections were rinsed in Tris-buffered saline (pH 7.6) with 0.1% Triton X-100, and blocked with 3% normal goat serum in phosphate buffered saline (PBS) with 1% bovine serum albumin and 0.1% Triton for 20 minutes at room temperature, followed by an avidin/biotin blocking step. Sections were then incubated with the primary antibody (rabbit anti-sEH, 1:10,000 (Draper and Hammock (1999) Toxicol. Sci., 50:30-5) overnight at 4° C. A secondary goat anti-rabbit biotinylated antibody was applied for 30 minutes at room temperature, and sections were then incubated with avidin-biotin-peroxidase complex (Vectastain Elite kit, Vector Laboratories, Burlingame, Calif., USA) for 30 minutes. The color reaction was visualized with diaminobenzidine and sections were lightly counterstained with Mayer's hematoxylin. Sections were dehydrated, overlaid with Permount, then coverslip, and observed with a light microscope. Negative controls were performed by replacing the primary antibody with nonimmune rabbit serum and by pre-absorbing the anti-sEH antibody with purified sEH protein.

### Isolation of Cerebral Microvessels

To determine the relative distribution of sEH protein in vascular versus non-vascular compartments, cerebral microvessels were separated from parenchymal brain tissue according to a published protocol (Ospina et al. (2002) Stroke 33:600-5) with slight modifications. Briefly, two mouse brains were pooled, homogenized in ice-cold PBS with a loosely fitting Dounce homogenizer, and centrifuged at 2,000 g for 5 minutes at 4° C. The supernatant was removed and stored on ice. The pellet was resuspended in PBS and centrifuged at 2,000 g for another 5 minutes at 4° C. The supernatant was combined with the first supernatant and centrifuged for 10 minutes at 3,000 g at 4° C. The resulting pellet containing the parenchymal fraction was stored at −80° C. The first pellet was resuspended in PBS, carefully layered over a 15% dextran density gradient (molecular weight 35,000 to 40,000 kDa), and centrifuged in a swinging-bucket rotor for 35 minutes at 3,500 g at 4° C. The supernatant was discarded, and the pellet was resuspended in PBS, layered over dextran and centrifuged for an additional 35 minutes at 3,500 g. The resulting pellet was thoroughly washed with ice-cold PBS over a 70 μm nylon mesh and cerebral vessels were collected and stored at −80° C.

### Protein Extraction, Sodium Dodecyl Sulfate-Polyacrylamide Gel Electrophoresis, and Western Blot

Brain tissue was homogenized in solution A containing sucrose (250 mmol/L), KCl (60 mmol/L), Tris-HCl (15 mmol/L), NaCl (15 mmol/L), ethylenediamine tetraacetic acid (5 mmol/L), ethylene glycol tetraacetic acid (1 mmol/L), phenylmethanesulfonyl fluoride (0.5 mmol/L), and dithiothreitol (10 mmol/L), then centrifuged at 2,000 g for 10 minutes at 4° C. to isolate cytoplasmic protein. Parenchymal pellets and vessels were processed in a similar manner, except that solution A was supplemented with 0.5% Triton X-100. Protein samples (15 μg) were separated by sodium dodecyl sulfate-polyacrylamide gel electrophoresis and transferred to polyvinylidene difluoride membranes. Blots were then blocked in 5% dry milk and incubated at 4° C. overnight with a primary rabbit anti-sEH antibody. Signal was visualized using a biotinylated secondary antibody (Amersham Biosciences, Piscataway, N.J., USA) with an ECL plus (Amersham) chemiluminescence detection kit. Autoradiograms were scanned and band optical densities quantified with QuantityOnes software (BioRad Laboratories, Hercules, Calif., USA). Blots were re-probed for the vascular smooth muscle cell marker α-actin (Chemicon International, Inc., Temecula, Calif., USA) and for β-actin (Sigma-Aldrich, St Louis, Mo., USA) to ensure equal loading.

### Soluble Epoxide Hydrolase Activity Assay

To determine if systemically administered AUDA-BE effectively suppresses enzyme activity in brain, sEH activity was determined at 1, 3, 6, and 24 hours after drug administration (two animals at each time point, n=8 total per group) using [³H]-trans-diphenylpropene oxide (tDPPO) as a substrate (Borhan et al. (1995) Anal. Biochem., 231:188-200). Rate of hydration of tDPPO is determined by liquid scintillation spectroscopy after differential extraction of the epoxide and diol. Briefly, cytosolic or peroxisomal fractions were incubated at 37° C. in 100 μL incubation mixtures containing sodium phosphate buffer (90 mmol/L, pH 7.4) and tDPPO (1 to 50 μmol/L in 1 μL dimethylformamide). Incubations were stopped after 5 minutes with the addition of 60 μL methanol and 200 μL isooctane. Zero-time and zero-protein incubations served as blanks. Incubations were vortexed vigorously to extract the substrate into the isooctane (the diol metabolite remains in the aqueous phase). A known aliquot of the aqueous phase was removed and added to 1 mL scintillation cocktail for scintillation counting. Extraction efficiency exceeded 91%.

Regional Cerebral Blood Flow [¹⁴C]iodoantipyrine Autoradiography

End-ischemic regional CBF was measured in a nonsurvival cohort of C57Blk/6 mice using quantitative autoradiography with [¹⁴C]IAP, as described previously (Alkayed et al. (2001) J. Neurosci., 21:7543-50). Two groups of mice (10 mg/kg AUDA-BE given 30 minutes before MCAO and vehicle, n=5 per group) were instrumented with femoral artery and jugular vein catheters, and MCA occluded as in the previous cohort. At 2 hours of MCA occlusion, 1 mCi of [¹⁴C]IAP in 75 μL of saline was infused intravenously for 45 seconds. Free-flowing arterial blood was simultaneously sampled at 5-second intervals for the arterial input function. With the filament in place, mice were decapitated at 45 seconds after the start of infusion, and the brain was quickly removed and frozen in 2-methylbutane on dry ice. Brains were sectioned on a cryostat into 20-mm-thick coronal slices, which were thaw-mounted on coverslips and then apposed to film (Kodak, BioMax MR, Eastman Kodak Company, Rochester, N.Y., USA) with ¹⁴C standards for 3 weeks. The concentration of [¹⁴C]IAP in blood was determined by liquid scintillation spectroscopy (Beckman 6500) after decolorization with 0.2 mL of tissue solubilizer (Soluene-350, PerkinElmer, Waltham, Mass., USA). Autoradiographic images representing five different coronal levels (+2, +1, 0, −1, and −2 mm from Bregma, 3 images each) were digitized, and regional CBF was determined in specific regions with the use of image analysis software (MCID, 7.0). Additionally, areas perfused with specific regional CBF rates were isolated by digital image scanning and summed to construct a histogram distribution of brain tissue over incremental ranges of blood flow rates. Areas were averaged among two images and were integrated across five coronal levels to calculate brain tissue volumes perfused with particular flow rates.

### Pharmacokinetic Parameters of Soluble Epoxide Hydrolase Inhibitors

12-(3-Adamantan-1-yl-ureido)-dodecanoic acid butyl ester was dissolved in sesame oil and administered as a single intraperitoneal injection of 10 or 40 mg/kg to C57Bl/6 mice (20 to 26 g). A second set of animals was injected with sesame oil alone (vehicle). Two mice from each treatment were decapitated; blood samples were collected at 1, 3, 6, and 24 hours after administration. 12-(3-Adamantan-1-yl-ureido)-dodecanoic acid butyl ester, its equally active metabolite 12-(3-adamantan-1-yl-ureido)-dodecanoic acid (AUDA) and the inactive metabolite 12-(3-adamantan-1-yl-ureido)-butyl acid (AUBA) were measured in blood samples as described previously (Morisseau et al. (2002) Biochem. Pharmacol., 63:1599-608; Watanabe et al. (2006) Anal. Chim. Acta., 559:37-44). Briefly, blood samples (10 μL) were collected in 1.5 mL Eppendorf microcentrifuge tubes, weighed, and vortexed with 100 μL of purified water. Samples were spiked with a surrogate compound (1-adamantyl-3-decyl urea, 790, 100 ng/mL) and extracted twice with 500 μL ethyl acetate, dried under nitrogen, and reconstituted in 25 μL of methanol containing 1-cyclohexyl-3-tetradecyl urea (500 ng/ml) as internal standard. Aliquots (10 μL) were analyzed by liquid chromatography coupled mass spectroscopy LC-MS/MS (Watanabe et al. (2006) Anal. Chim. Acta., 559:37-44; Watanabe and Hammock (2001) Anal. Biochem., 299:227-34).

The concentration of sEH inhibitors were quantified in extracts using HPLC with positive mode electrospray ionization and tandem mass spectrometry. Analytes were quantified on a 5-point curve with internal standard methods. Surrogate recoveries were evaluated by quantification against the internal standard. Surrogate recoveries were 70% for all reported plasma samples, the analysis of reagent blanks, matrix spikes, and analytical replicates were used to document method stability during this study. Molecular ion and transition ion for sEH inhibitors were as follows: AUDA-BE (449.2>272.2), AUDA (393.1>135), AUBA (281.2>104). Transition ions for the surrogate compound 790 was 335.1>135 and internal standard 1-cyclohexyl-3-tetradecyl urea was 341.2>216.2.

### Statistical Analysis

Differences in infarct size, enzyme activity, and blood flow rates were analyzed with a t-test for two groups and analysis of variance with post hoc Newman-Keuls multiple range test for multiple groups. The pharmacokinetic profile was examined for goodness of fit to a noncompartmental or multicompartmental models. Pharmacokinetic parameters were then obtained by fitting the blood-concentration-time data to a non-compartmental model with the WinNonlin software (Pharsight, Mountain View, Calif., USA), as described previously (Watanabe et al. (2006) Anal. Chim. Acta., 559:37-44). The criterion for statistical significance was set at P<0.05. All values are reported as mean±s.e.m.

### Results

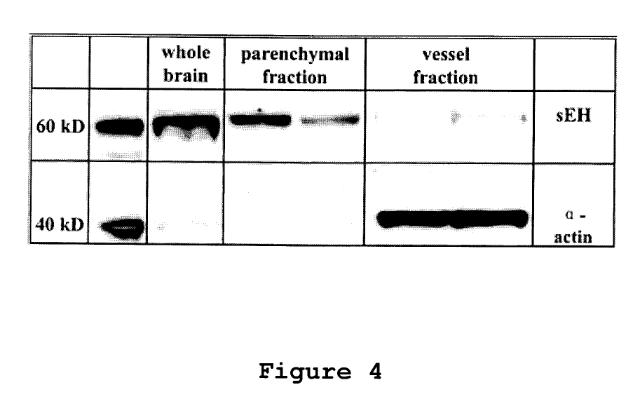

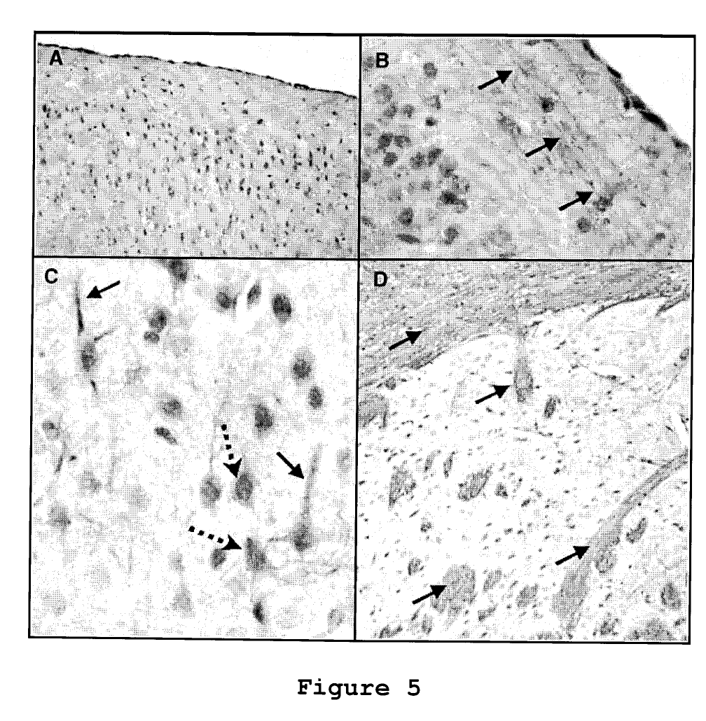

To determine if sEH is differentially expressed in cerebral blood vessels and brain parenchyma, brain tissue was fractionated into vascular and nonvascular compartments and both compartments probed with anti-sEH antibody. FIG. 4 shows that sEH immunoreactivity (62.5 kDa) is found in both compartments. However, in brain, sEH is predominantly localized in the parenchymal fraction, and to a lesser extent in the vascular compartment, as identified by the vascular smooth muscle marker α-actin (40 kDa). This observation was further examined using immunohistochemistry. FIG. 5 shows that sEH is abundantly expressed in brain, especially in areas relevant to the stroke model used in the current study, such as the cerebral cortex (FIG. 5A), and striatum (FIG. 5D). Immunoreactivity was predominantly localized in neurons (arrows in FIG. 5C), and it did not co-localize with the astrocyte marker glial fibrillary acidic protein (GFAP). Within neurons, sEH immunoreactivity was observed in neuronal cell bodies (dotted arrows in FIG. 5C) and dendrites (solid arrows in FIG. 5C). The most striking finding, however, was the localization of sEH immunoreactivity in axons (fine intersecting processes within neuropil in FIG. 5C) and nerve fiber bundles within gray (arrows in FIGS. 5B and 5D) and white matter (upper arrow in FIG. 5D). Notably, in the striatum (FIG. 5D), immunoreactivity was not observed in neuronal cell bodies and was primarily localized in fiber bundles (cross-sections in FIG. 5D). Less consistent immunoreactivity was seen in vascular cells and in cells morphologically consistent with microglia. No staining was observed when the primary antibody was removed, and pre-absorbing anti-sEH antibody with purified sEH protein completely eliminated staining.

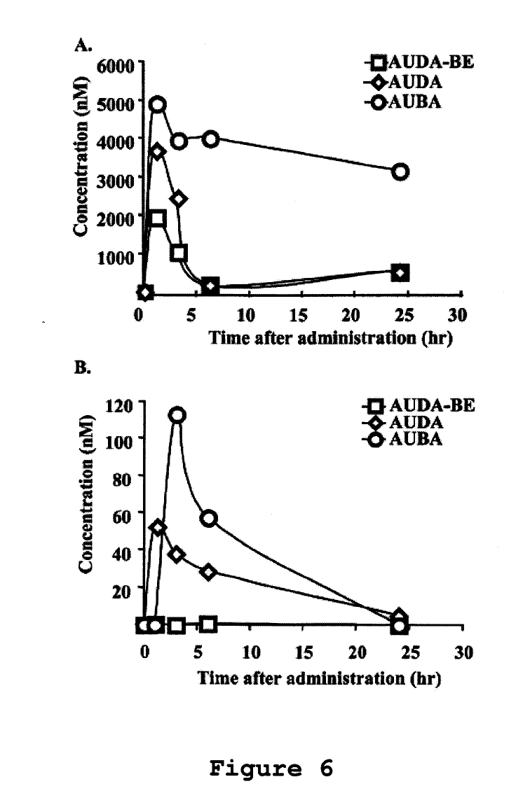

To determine blood concentrations of AUDA-BE and its active metabolite AUDA as well as its inactive indicator metabolite (AUBA) pharmacokinetic studies were performed after a single intraperitoneal injection of 10 and 40 mg/kg AUDA-BE. A time-dependent analysis of plasma sEH inhibitor levels is summarized in FIG. 6. 12-(3-Adamantan-1-yl-ureido)-dodecanoic acid butyl ester reached a maximum concentration of 2 μmol/L within the first hour after 40 mg/kg injection (FIG. 6A). However, when 10 mg/kg AUDA-BE was injected, only the metabolites AUDA and AUBA were detected (FIG. 6B). 12-(3-Adamantan-1-yl-ureido)-dodecanoic acid butyl ester was rapidly hydrolyzed to AUDA and reached maximum concentration of 4 μmol/L within the first hour (FIG. 6A). AUDA and AUDA-BE were further degraded to AUBA. 12-(3-Adamantan-1-yl-ureido)-butyl acid (5 μmol/L) was observed in the first hour and the concentration remained relatively stable at 3.5 μmol/L for 24 hours after injection (FIG. 6A). FIG. 6B shows that, when administered at 10 mg/kg, AUDA-BE is rapidly metabolized, and that its active metabolite AUDA reaches a peak concentration of 52 nmol/L at 1 hour, with a slow degradation rate over 24 hours, whereas AUBA reaches a higher peak of 113 nmol/L at 3 hours, followed by a faster degradation rate over 24 hours. No apparent adverse effects were noted for either 10 or 40 mg/kg doses during the 24-hour period after drug administration. As the 10-mg/kg dose gives adequate blood levels and clear one-compartment pharmacokinetics, subsequent biological studies were performed with this dose. Pharmacokinetic parameters of AUDA in plasma show a maximum concentration of AUDA at 52 nmol/L within 1 hour, a half time of elimination of 7 hours and a mean residence time of 9 hours.

It has been previously shown that IC₅₀ values of AUDA-BE and AUDA are similar and are in the low nanomolar range (Kim et al. (2007) Bioorg. Med. Chem., 15:312-23; Morisseau et al. (2002) Biochem. Pharmacol., 63:1599-608). This concentration was also biologically effective in lowering blood pressure in hypertensive model (Imig et al. (2005) Hypertension 46:975-81). Therefore, in these experiments, the levels of sEH inhibitors in the blood are in the concentration range expected to inhibit the enzyme. To determine if AUDA-BE and its active metabolite cross the blood-brain barrier and reach adequate inhibitory concentrations, AUDA-BE and its metabolites were measured in brain tissue extracted with methanol and chloroform (Folch et al. (1951) J. Biol. Chem., 191:833-41). Although the variability of the inhibitor concentrations was high and the recovery of the compounds was low, significant levels of the active metabolite AUDA, but not the prodrug AUDA-BE or the inactive metabolite AUBA, were detected in homogenates of brain tissue. AUDA reached its maximum concentration within 3 hours after a single injection of 10 mg/kg AUDA-BE with a mean residence time of 6 hours and half-time elimination of 4 hours.

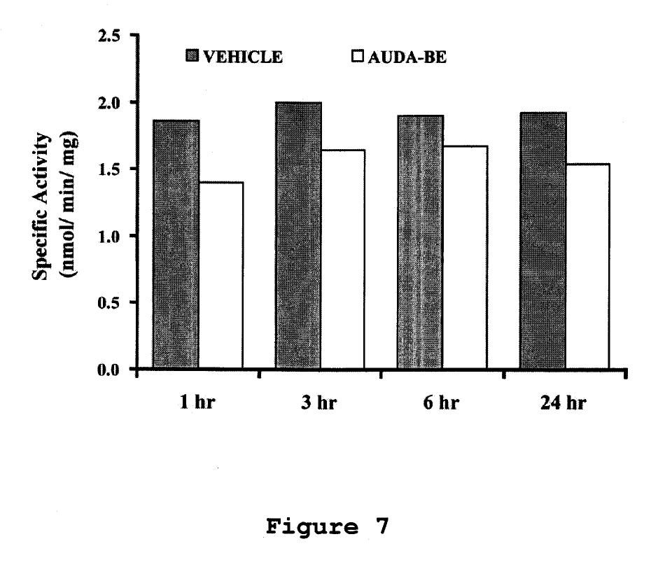

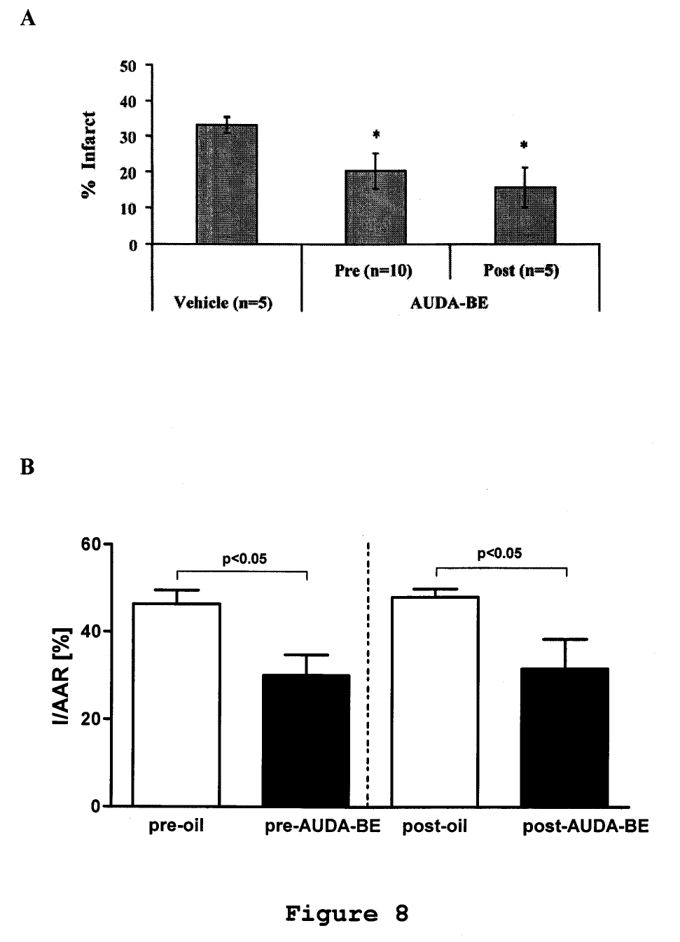

To determine if these concentrations produce adequate inhibition of enzyme activity in brain, sEH hydrolase activity was measured in brain at 1, 3, 6, and 24 hours after AUDA-BE administration (10 mg/kg intraperitoneally, n=2 per group at each time point) using sEH-specific substrate [³H]-trans-diphenylpropene oxide (tDPPO). Brain sEH activity was significantly inhibited by AUDA-BE, and at least 20% inhibition persisted for up to 24 hours (FIG. 7). It should be noted that these sEH inhibitors are reversible transition state inhibitors. Thus, the levels of inhibition are certain to be above those detected due to dilution of the sample during work up. Having established the effectiveness and bioavailability of AUDA-BE in brain tissue, its effect on ischemic brain injury was determined. FIG. 8A summarizes the effect of AUDA-BE on infarct size after MCAO in mice. Compared with vehicle, AUDA-BE (10 mg/kg) reduced infarct size after 2-hours MCAO by 40 and 50% when administered 1 hour before (pre, n=5 per group) or at the time of reperfusion (post, n=5 per group), respectively (P<0.05). FIG. 8B demonstrates infarct size as a percent of area at risk (I/AAR).

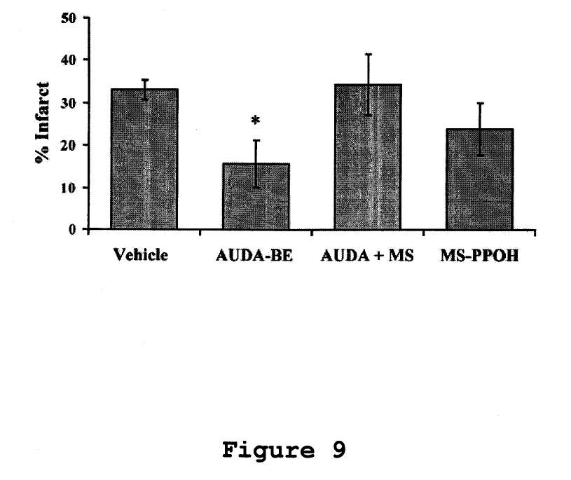

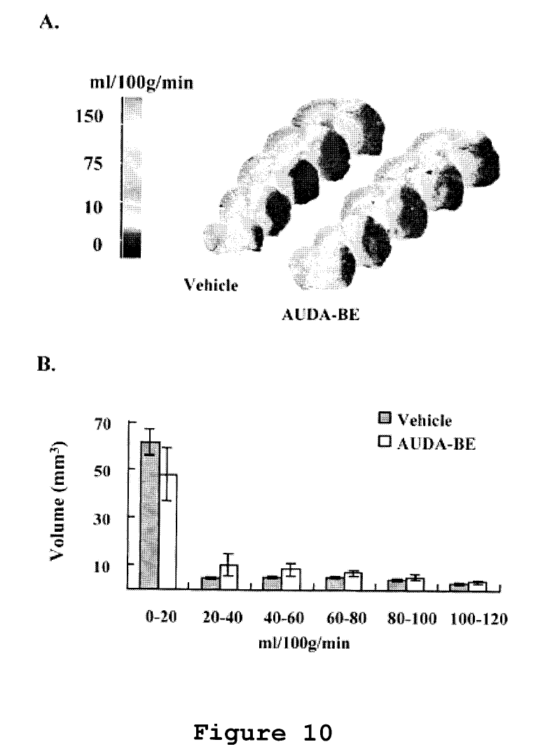

To determine if the protection observed by sEH inactivation is linked to EETs, AUDA-BE was co-administered with MS-PPOH, which inhibits EETs biosynthesis via the P450 epoxygenase pathway (Brand-Schieber et al. (2000) J. Physiol. Pharmacol., 51:655-72). To ensure adequate inhibition of EETs synthesis and depletion of already formed EETs, MS-PPOH (0.5 mg/200 μL) was administered over 24 hours before MCAO. 12-(3-Adamantan-1-yl-ureido)-dodecanoic acid butyl ester was administered as a single intraperitoneal injection (10 mg/kg) at the time of reperfusion. FIG. 9 shows that MS-PPOH blocks infarct size reduction by AUDA-BE. When administered alone, AUDA-BE reduces infarct size from 33±2% to 16±6% (n=5 per group, P<0.05). However, when combined with MS-PPOH, AUDA-BE loses its protective effect (infarct size 34±7%, n=5, which is not different from vehicle), which is not attributable to a nonspecific effect of MS-PPOH, because MS-PPOH alone (24±6%, n=5) does not increase infarct size after MCAO.

[¹⁴C] IAP autoradiography was then used to determine if AUDA-BE alters CBF during vascular occlusion. FIG. 10A is color-coded distribution of CBF in ischemic and contralateral hemispheres, and FIG. 10B is histogram distribution of brain tissue volume over 20 mL/100 g per min increments of CBF rates. FIG. 10B shows that regional CBF distribution within the ipsilateral hemisphere at the end of 2-hour MCAO was not different between AUDA-BE and vehicle-treated animals (n=5 per group), suggesting that protection by AUDA-BE is not related to altered ischemic severity.

Accordingly, the above demonstrates that (1) sEH is expressed at high levels and is highly active in rodent brain, (2) the predominant localization of sEH in brain is in neuronal cell bodies and processes, and to a lesser degree in cerebral blood vessels, (3) inhibition of brain sEH is protective against ischemic brain damage, (4) the mechanism of protection is linked to the P450 epoxygenase, but it is not mediated through increased CBF during vascular occlusion. These findings implicate sEH as an important player in ischemic brain injury and thus this molecule serves as a novel therapeutic target in stroke. Additionally, it was determined that the potent sEH inhibitor AUDA-BE or its active metabolite, AUDA, is capable of crossing blood-brain barrier and effectively suppressing brain sEH enzymatic activity.

### EXAMPLE 3

### Materials and Methods

The study was conducted in accordance with the National Institute of Health guidelines for the care and use of animals in research and protocols were approved by the institutional animal care and use committee. The sEHKO mice were obtained from Dr. Frank Gonzalez at the National Institutes of Health, where it was originated. The gene disruption strategy used and the phenotype of the sEHKO mice are described in Sinal et al. (J. Biol. Chem. (2000) 275:40504-4051). The strain has been backcrossed to C57Bl/6 for at least six generations and, therefore, homozygous sEHKO mice were compared to wild-type (WT) C57BL/6 mice obtained from The Jackson Laboratories (Bar Harbor, Me.). Homozygous sEHKO mice are viable, fertile, normal in size and do not display any gross physical or behavioral abnormalities. The mouse genotype was confirmed by PCR as previously described (Sinal et al. (2000) J. Biol. Chem., 275:40504-4051).

Adult male C57BL/6 mice (WT, 6-8 weeks of age, n=15) and age- and weight-matched sEHKO mice were subjected to cardiac arrest (CA). In random order with respect to their strain, mice were removed from their home cages, and anesthesia was induced with 4% isoflurane, and subsequently maintained with 1-2% isoflurane in air/oxygen mixture. Mice were weighed, positioned on the operating and mechanically ventilated after endrotracheal intubation with a 22 gauge catheter. Body temperature was monitored with a rectal probe and maintained at 37±0.5° C. with a heating lamp and warm pad.

Using sterile technique, a PE-10 catheter was inserted into the right jugular vein for intravenous (i.v.) drug infusion. Electrocardiogram (EKG) was monitored with subdermal electrodes, and arterial blood pressure was monitored using a femoral arterial catheter in a cohort of 6 animals (3 WT and 3 sEHKO mice). CA was induced with 40 μL iced 0.5M KCl i.v. and confirmed by EKG and, when present, arterial blood pressure measurement. Ventilation was stopped and the endrotracheal tube disconnected from the ventilator. After 9 minutes and 30 seconds of normothermic CA with no ventilation, the endrotracheal tube was reconnected to the ventilator, and hyperventilation at 120% of pre-arrest rate was initiated using 100% O₂. At 10 minutes, chest compressions were initiated at a rate of 300 bpm, and epinephrine (8-15 mcg in 0.5-1 mL normal saline) was administered intravenously in divided doses. CPR was discontinued upon return of spontaneous circulation (ROSC) as observed on the EKG, or when 4 minutes of CPR had passed without ROSC. EKG and temperature were closely monitored for 10 minutes after ROSC. Animals were extubated when spontaneous respiratory rate was greater than 60/minute, usually between 12 and 18 minutes after ROSC. The jugular catheter was removed, hemostasis obtained, and animals returned to cages. The recovery cage was placed on a warming mat set at 37° C. to maintain normothermia in the post arrest period. Animals were intermittently observed over the 24 hours following for survival and activity. Surviving animals were deeply anesthetized at 24 hours after CA, and tissue fixed through transcardiac perfusion with 4% paraformaldehyde in saline for subsequent histological analysis.

### Results

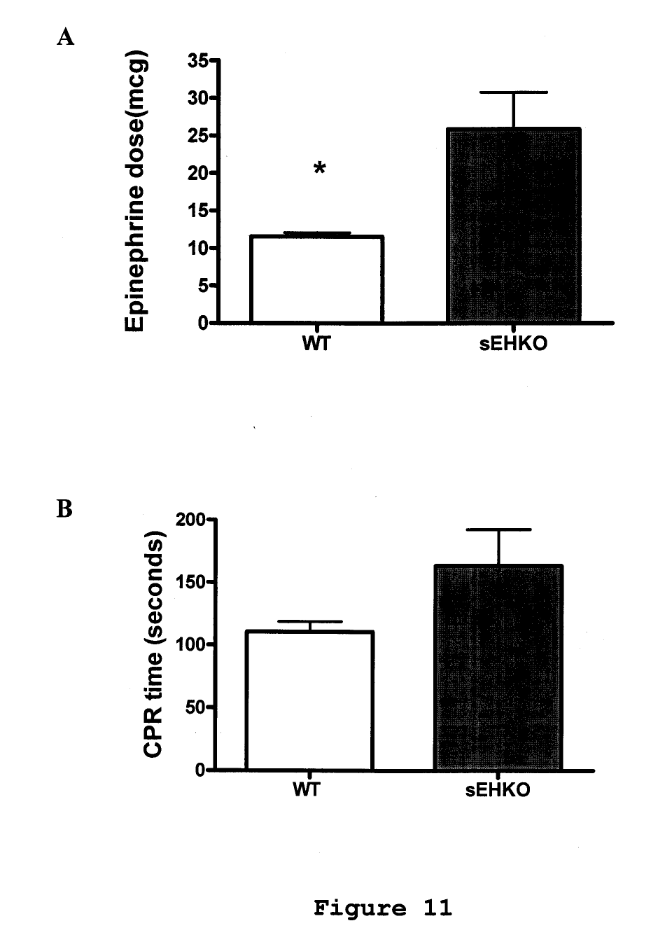

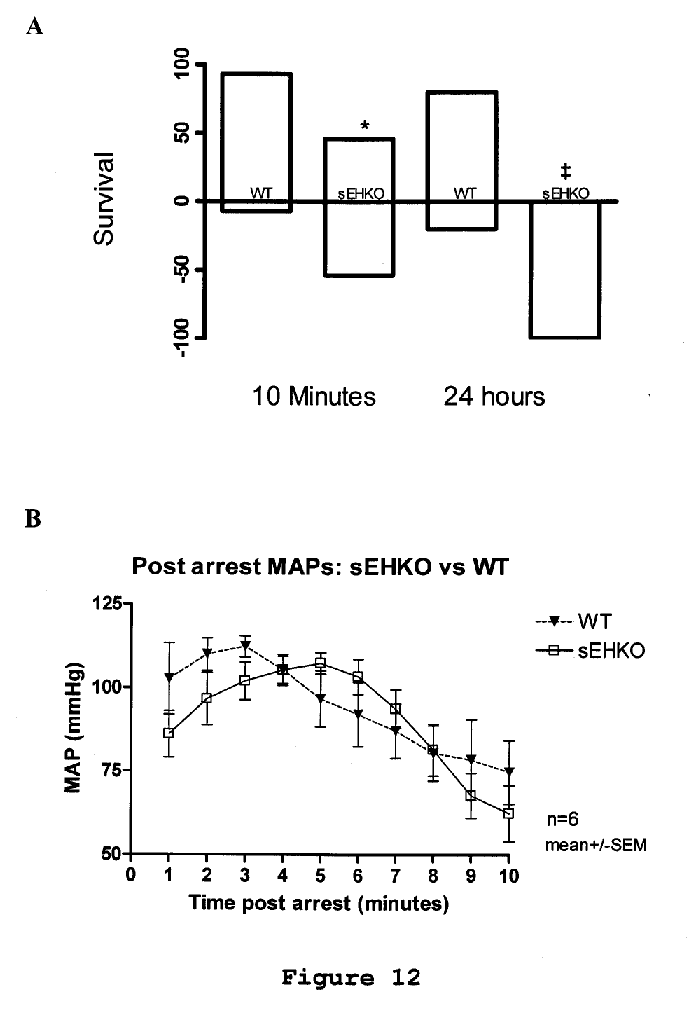

There were no significant differences between WT and sEHKO mice with regard to pre-arrest body weight, baseline or mean intra-arrest rectal temperature (Table 2, n=15 per group). Both strains of mice were subjected to identical protocol of 10-minute normothermic cardiac arrest, followed by cardiopulmonary resuscitation (CPR) under isoflurane anesthesia. In WT mice, restoration of spontaneous circulation (ROSC) required 12.0±0.5 mcg of epinephrine and 110.3±8.2 seconds of CPR, resulting in 93% and 80% survival at 10 min and 24 hours after CA/CPR (14 and 12 survivors out of 15 mice, respectively). Unexpectedly, sEHKO mice were refractory to resuscitation from cardiac arrest. They required significantly more epinephrine (26.0±4.9 mcg, n=12, p=0.003, FIG. 11A), and longer CPR time (163.3±28.9 seconds, n=12, p=0.06, FIG. 11B) compared to WT mice. More importantly, survival was markedly lower in sEHKO mice compared to WT mice. Only 46% of sEHKO mice survived for 10 minutes (7 out of 15) and no sEHKO mice survived 24 hours after CA/CPR (n=15, FIG. 12A). Most of the 10-minute survivors expired within 2 hours post arrest. In a separate cohort of WT and sEHKO mice, mean arterial blood pressure (MAP) was monitored during cardiac arrest and for 10 minutes after resuscitation (FIG. 12B). In WT mice, MAP recovered to 103±11 mmHg by 1 minute, peaked at 112±3 mmHg by 3 minutes, and reached 75±10 at 10 minutes after CA/CPR. Arterial blood pressure recovery was delayed in sEHKO mice, reaching 86±7 mmHg at 1 minute, peaking at 107±3 mmHg by 5 minutes, and dropping to 62±8 mmHg at 10 minutes after CA/CPR.

TABLE 2
Baseline and intra-arrest temperatures in WT and sEHKO mice.
There were no significant differences in body weight, baseline
or intra-arrest temperature between the two strains.

| | | Baseline | Mean arrest |
|---|---|---|---|
| | Weight | temperature | temperature |
| | (gms, mean ± | (° C., mean ± | (° C., mean ± |
| Strain | SEM, n = 15/gr) | SEM, n = 15/gr) | SEM, n = 15/gr) |
| WT | 24.1 ± 1.05 | 36.6 ± 0.16 | 37.0 ± 0.02 |
| sEHKO | 21.5 ± 0.64 | 36.0 ± 0.22 | 36.7 ± 0.21 |

Accordingly, it is evident that the deletion of the sEH gene deletion renders mice refractory to cardiopulmonary resuscitation (CPR) after cardiac arrest (CA). The SEHKO mice required significantly higher doses of epinephrine and longer CPR time, demonstrated delayed blood pressure recovery after CPR and suffered significantly higher mortality compared to wild-type control mice. These findings indicate that sEH plays an important role in recovery from cardiac arrest, likely due to its EETs-metabolizing function.

Without being bound by theory, the cause for the increased mortality in sEHKO mice may be the slower recovery in arterial blood pressure recovery in sEHKO mice, thereby requiring higher doses of epinephrine and longer CPR time for recovery. As mentioned above, EETs are vasodilators and sEHKO mice are resistant to angiotensin- and salt load-induced hypertension (Sinal et al. (2000) J. Biol. Chem., 275:40504-40510). Therefore, sEH gene disruption may confer either a baseline state of increased arteriolar vasodilation relative to wild type, a resistance to the effects of exogenous vasoconstrictors, or both. The concept that baseline arteriolar tone is reduced in sEHKO mice is supported by the observation that sEHKO mice have mean systolic blood pressures 13 mmHg lower than WT controls (Sinal et al. (2000) J. Biol. Chem., 275:40504-40510), but do not have reduced fractional shortening or other indices of myocardial function (Seubert et al. (2006) Circ Res., 99:442-450). Notably, sEHKO mice have been shown to have similar cardiac mass as WT mice (Seubert et al. (2006) Circ Res., 99:442-450). Therefore, it is unlikely that higher sEHKO mortality is attributed to a primary cardiac cause. However, increased mortality in sEHKO mice may be related to other yet unrecognized effects of EETs. For example, sEH inhibition has been shown to increase pulmonary vascular resistance (Pokreisz et al. (2006) Hypertension 47:762-770), which, if it takes place in sEHKO mice after CA, would lead to pulmonary hypertension and right ventricular failure, and would impede resuscitation.

### EXAMPLE 4

The arachidonic acid (AA) derivates epoxyeicosatrienoic acids (EETS) have anti-inflammatory and anti-thrombotic effects, are coronary vasodilators, and reduce myocardial ischemic injury. The biological activity of EETs is terminated by hydration into less active dihydroeicosatrienoic acids (DHETs) by soluble epoxide hydrolase (sEH). More than 10 genetic variants of the EPHX2 gene encoding soluble epoxide hydrolase in humans have been identified and these polymorphisms have been implicated in susceptibility to cardiovascular disease (Fornage et al. (2005) Hum. Mol. Genet., 14:2829-2837; Lee et al. (2006) Hum. Mol. Genet., 15:1640-1649). Experimental studies showed that these variants influence the activity of the resulting sEH and influence neuronal survival in vitro (Przybyla-Zawislak et al. (2003) Mol. Pharmacol., 64:482-490; Koerner et al. (2007) J. Neurosci., 27:4642-4649).

As stated hereinabove, epoxyeicosatrienoic acids (EETs) protect against myocardial ischemic injury in vivo. The hypothesis that EPHX2 polymorphisms alter cardiomyocyte survival after simulated ischemia was tested.

Animals were allowed access to phytoestrogen-free food and water ad libitum. With local IACUC approval, all animals received treatment in compliance with the Guide for the Care and Use of Laboratory Animals (Institute of Laboratory Animal Research, National Research Council; National Academy Press, 1996).

Post natal Day 7-8 C57BL\6 mice were used. 3-4 hearts were obtained from same-gender mice. Cells were dissociated with 0.625% wt/volume trypsin in Ca²⁺ and Mg²⁺ free HBSS. The cells were centrifuged and re-suspended in FBS-M199. Cells were supplemented with estrogen-free 15% FBS, 100 U/ml penicillin, 100 μg/ml streptomycin, and 25 μM cytosine arabinoside. The cells were then plated at 37° C. for 1 hour under a water-saturated atmosphere of 5% CO₂-95% O₂. Suspended cells were collected and plated at 1.0×10⁵ cells/cm² and incubated as above for 24 hours.

Six TAT-hsEH fusion proteins, including one wild type and five EPHX2 mutants (Lys55Arg, Arg103Cys, Cys154Tyr, Arg287Gln, and Arg103Cys/Arg287Gln), were expressed and purified as reported (Koerner et al. (2007) J. Neurosci., 27:4642-4649).

TAT-hsEH fusion proteins were added to the medium of cultured cardiomyocytes to the final concentration of 100 nM. The cells were incubated at 37° C. for 24 hours before the onset of oxygen-glucose deprivation.

For oxygen-glucose deprivation (OGD), cells were placed in a Plexiglas hypoxia chamber under 100% N₂ at 37° C. and glucose-free medium (MEM/HBSS). For reoxygenation, cells were placed in 21% O₂ (5% CO₂) and glucose-replete FBS-M199 medium.

Cell death was assessed via propidium iodide (PI, 5 μM) with >300 cells/sample.

For Western blot analysis, 4-20% linear gradient SDS-polyacrylamide gels (Bio-Rad, Hercules, Calif.) in a mini gel apparatus (Mini-PROTEAN3®, Bio-Rad) were used. Blots were incubated overnight at 4° C. with primary rabbit anti-sEH antibody (1:2000 in 5% dry milk, Santa Cruz, Santa Cruz, Calif.). Detection was performed via a luminescence method (ECL-plus Western blotting detection kit, Amersham) with peroxidase-linked anti-rabbit (1:2000 in 5% dry milk).

For 14,15-DHET ELISAs, TAT-hsEH (wild-type; Lys55Arg; Arg287Gln) transduced cardiomyocytes were spiked with 14,15-EET (1 μM) for 4.5 hours. 14,15 DHET production was measured with 14,15-DHET immunoassay ELISA.

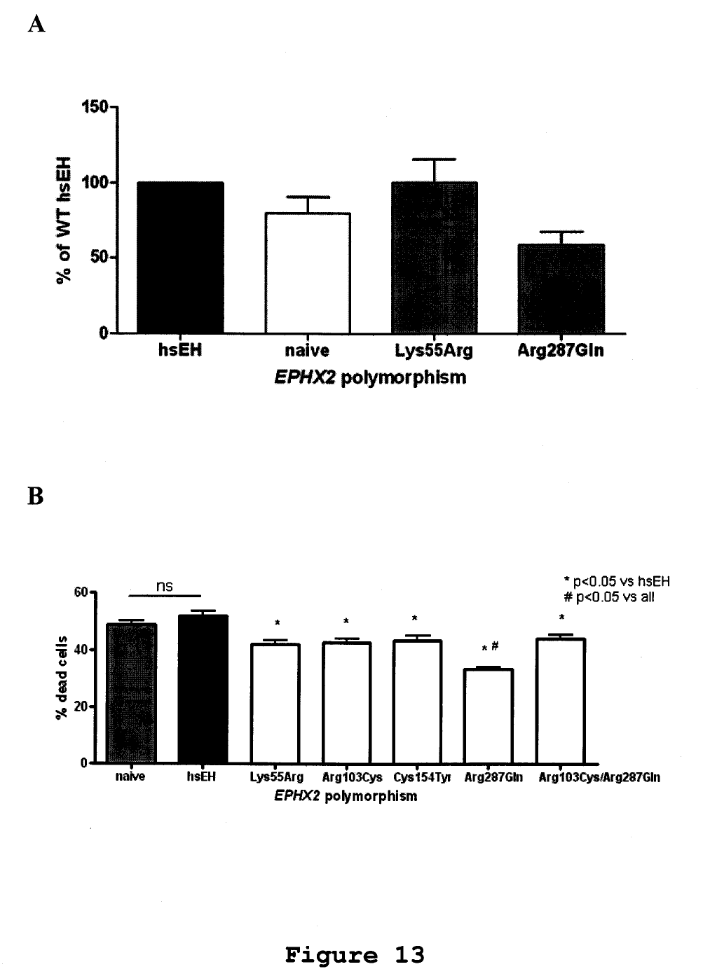

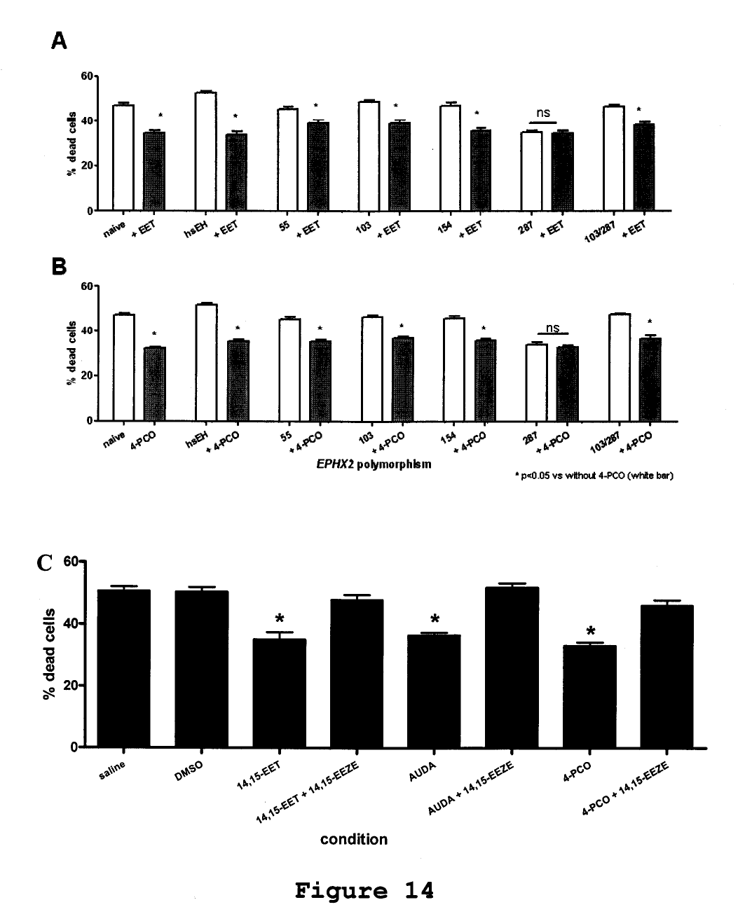

The Arg287Gln mutation exhibits reduced hydrolase activity (FIG. 13A). Cultured neonatal murine cardiomyocytes were transduced with five human EPHX2 variants linked to the protein transduction domain TAT. Cultures were subjected to 1.5 hours of oxygen and glucose deprivation followed by 3 hours of re-oxygenation, with or without sEH substrate 14,15-EET. Cell death was assessed by propidium iodide (% of all cells, mean±SEM, n=5-9 replicates). Successful transduction was confirmed by immunoblot. All mutants reduced cell death compared to the human wild-type EPHX2 (rh-sEHWT, 63±1%), but only the Arg287Gln mutations was different from untransduced controls (FIG. 13B). Excess 14,15-EET improved cell survival in all polymorphisms tested except for Arg287Gln (45±1% vs. 45±1%) and the inhibition of sEH by 4-phenylchalcone oxide (4-PCO) also improved cell survival significantly in all groups tested except for the Arg287Gln (FIG. 14). These data explain genetic variability insensitivity to ischemic injury. Further, FIG. 14C demonstrates that the ability of sEH inhibition to increase cardiomyocyte viability after OGD is abolished by 14,15-EET antagonists.

### EXAMPLE 5

As stated hereinabove, the P450 epoxygenase pathway metabolizes arachidonic acid (AA) into 4 biologically active eicosanoids referred to as epoxyeicosatrienoic acids (5,6-, 8,9-, 11,12-, and 14,15-EET) (Liu et al. (2004) Curr. Drug Metab., 5:225-234). EETs play an important role in regulating tissue perfusion in both cardiac and extra cardiac organs. The actions of EETs are terminated by conversion to dihydroxyeicosatrienoic acids (DHETs) by epoxide hydrolases (Morisseau et al. (2005) Annu. Rev. Pharmacol. Toxicol., 45:311-333). Two major epoxide hydrolases are found in mammalian tissues, the microsomal (mEH) and soluble (sEH) epoxide hydrolases (Fang et al. (2004) Am. J. Physiol. Heart Circ. Physiol., 287:H2412-H2420). However, sEH is the primary enzyme involved in the in vivo metabolism of EETs (Zeldin et al. (1993) J. Biol. Chem., 268:6402-6407). In addition to their vascular effects, EETs exhibit a cardioprotective effect, which has been linked to activation of the RISK pathway (Yellon et al. (2007) N. Engl. J. Med., 357:1121-1135), and mediated in part through activation of the phosphatidyl-inosiltol-3-kinsae (PI3K)/Akt pathway and the mitochondrial KATP channels (Seubert et al. (2004) Circ. Res., 95:506-514; Gross et al. (2007) J. Mol. Cell Cardiol., 42:687-691). Augmenting endogenously released EETs by inhibiting the converting enzyme sEH represents an attractive strategy to increase ischemic tolerance. In the current studies, the effects of both sEH inhibition and gene deletion on cardiac injury were examined using a clinically relevant in vivo model of regional myocardial-ischemia-reperfusion (I/R). The findings presented herein demonstrate for the first time that: 1) a selective sEH inhibitor is cardioprotective when administered either before or at the end of regional myocardial ischemia, 2) mice with sEH gene deletion sustain smaller damage after in vivo myocardial I/R compared to wild-type control mice, and 3) exogenously administered sEH substrate 14,15-EET reduces infract size after myocardial I/R, whether administered before or at the end of the ischemic period. Finally, the first immunohistochemical evidence of sEH localization in cardiac myocytes of left ventricular tissue is provided.

### Materials and Methods

### Animals

The Institutional Animal Care and Use Committee at the Portland VA Medical Center approved this study, and all animals received treatment in compliance with the Guide for the Care and Use of Laboratory Animals (Institute of Laboratory Animal Research, National Research Council; National Academy Press, 1996). Study animals were allowed access to phytoestrogen-free food (#2014, Harlan Teklad, Madison, Wis.) and water ad libitum until induction of anesthesia.

A colony of sEHKO mice with targeted deletion of the EPHX2 gene is maintained in-house. These mice have been backcrossed to C57BL/6 for at least seven generations, and therefore, homozygous sEHKO mice were compared to wild-type (WT) C57BL/6 mice obtained from The Jackson Laboratories (Bar Harbor, Me.).

### Regional Myocardial Ischemia-Reperfusion

Male mice between the ages of 16-24 weeks were anesthetized with isoflurane (induction 4-5 vol %; maintenance 1-2 vol %), intubated with a 20G plastic IV catheter, and mechanically ventilated using a rodent ventilator (Inspira, volume-controlled, Harvard Apparatus, MA). ECG and rectal temperature were continuously monitored using a PC-based recording device (PowerLab 8/30, ADInstruments, Colorado Springs, Colo.). The animals were positioned in a right lateral decubital position on a heating pad and rectal temperature was maintained at 37° C. throughout the experiment. A PE-10 catheter was inserted into the left jugular vein for intravenous drug infusion. Using a dissecting microscope, a left-sided thoracotomy was performed in the 4th intercostal space. Heparin (1 unit/gram body weight intraperitoneal) was given to prevent clotting during temporary occlusion of the left coronary artery (LCA) in all groups except for the pre-ischemia 14,15-EET group (which experienced excessive bleeding in pilot studies following heparin administration). A ligature (8-0 Monosof MV-135-4, ⅜ 5 mm tapered needle, Syneture, Norwalk, Colo.) was placed around the left coronary artery (LCA) approximately 2 mm distal to the left atrial margin. The suture was fed through a short section of PE-10 tubing to form a snare to allow temporary occlusion. The LCA was occluded for 40 minutes; occlusion was confirmed by persistent ECG changes during occlusion and visual paling of the left ventricle (LV). After 40 minutes the snare was released and reperfusion was confirmed with visual hyperemia of the LV and return of the ECG changes. After 2 hours of reperfusion the LCA was re-occluded and fluorescent polymer microspheres (0.4% solution, diameter 1-10 μm; Duke Scientific Corporation, Palo Alto, Calif.) were infused via needle puncture of the LV apex. The microspheres showed a maximum excitation at 360 nm long UV light and delineated the non-perfused, ischemic area-at-risk (AAR) as a negative image. Heart was then excised, rinsed in normal saline, and the right and left atria were removed under the dissecting microscope. The remaining left and right ventricles were weighed and then sliced into seven sections (d=1 mm) for imaging and staining.

### Measurement of AAR and Infarct Size

Heart sections were immediately photographed with a digital camera (Canon Powershot A620) under UV light for determination of AAR. Infarct size (I) was determined by staining the heart sections in 1% 2,3,5 triphenyltetrazolium chloride (TTC) in phosphate buffer at pH 7.4 for 10 minutes, followed by 10% neutral buffered formalin (NBF) bath overnight. Pictures were taken the next day under full spectrum light. TTC stained non-infarcted myocardium red, while infarcted myocardium remained white. Both AAR and I areas were calculated in a blinded fashion using standard imaging software (Photoshop Elements 4). A calibration ruler was present in each picture to convert pixels into volume. TTC slices were volume corrected for dehydration following formalin immersion.

### Drugs

The 14,15-EET was obtained from BIOMOL (Plymouth Meeting, Pa.). It was dissolved in a vehicle composed of 100% ethanol and saline (1:3), resulting in a final ethanol solution of 25%. The selective sEH inhibitor 12-(3-adamantan-1-yl-ureido)-dodecanoic acid butyl ester (AUDA-BE) was dissolved in sesame oil, as previously described (Zhang et al. (2007) J. Cereb. Blood Flow Metab., 27:1931-1940). 14,15-EET (2.5 μg/g body weight iv) or AUDA-BE (10 μg/g ip) was given either prior to LCA occlusion (PRE) or prior to reperfusion (POST) and compared to vehicle control. The experimental protocol and time line was as follows: vehicle or AUDA-BE was added 30 minutes prior to occlusion (which begins 40 minute ischemia period); SNC-162, vehicle, or 14,15-EET was added 15 minutes prior to occlusion; vehicle or AUDA-BE was added 10 minutes prior to reperfusion (i.e. LCA release); or vehicle or 14,15-EET was added 5 minutes prior to reperfusion. The AUDA-BE concentration used was based on the pharmacokinetic studies (Zhang et al. (2007) J. Cereb. Blood Flow Metab., 27:1931-1940). δ-opioid receptor agonists produce cardioprotection against myocardial I/R (Schultz et al. (2001) Pharmacol. Ther., 89:123-137). SNC-162, a non-peptide δ-opioid receptor agonist, was used for comparison of the magnitude of cardioprotection found within the epoxide pathway. SNC-162 (0.1 μg/g iv over 10 minutes) was dissolved in 10% DMSO.

### Immunoblotting

Proteins from left ventricular tissue were dissolved in SDS sample buffer (2% SDS, 10% glycerol, 80 mM Tris, pH 6.8, 0.15 M β-mercaptoethanol, 0.02% bromphenol blue) and separated on 4-20% linear gradient SDS-polyacrylamide gels (Bio-Rad) in a minigel apparatus (Mini-PROTEAN® 3, Bio-Rad) and transferred to polyvinylidene difluoride membranes. Membranes were blocked with 5% nonfat dry milk in TBST (10 mM Tris, pH 7.5, 150 mM NaCl, 0.05% Tween 20) for 60 minutes at room temperature and incubated overnight at 4° C. with primary rabbit anti-sEH antibody (1:2000 in 5% dry milk, Santa Cruz, Santa Cruz, Calif.), as described (Zhang et al. (2007) J. Cereb. Blood Flow Metab., 27:1931-1940). The antigens were detected by the chemiluminescence method (ECL-plus Western blotting detection kit, Amersham) with peroxidase-linked anti-rabbit secondary antibody (1:2000 in 5% dry milk). After immunoblotting, the intensity of immunoblot bands was detected with a Kodak Image Analysis System.

### Immunohistochemistry

Mouse left ventricular myocardial samples were fixed with fresh 4% paraformaldehyde, embedded in paraffin and cut into 5-μm sections. The sections were mounted on superfrost glass slides. Sections were kept at 60° C. overnight and then de-paraffinized with xylene followed by washing in 100%, 96%, 80%, and 70% ethanol. Heat unmasking of epitopes was done by boiling the samples three times in citrate buffer (pH-6) at 600 W in a microwave oven. After cooling down to room temperature and 1 hour of blocking in 5% dry milk, slides were incubated overnight at room temperature with primary antibodies (rabbit anti-sEH antibody, 1:50), as described (Iliff et al. (2007) Exp. Physiol., 92:653-658). After washing in PBS, sections were incubated with secondary FITC-conjugated goat anti-rabbit IgG (1:100 in 5% dry milk, Invitrogen, Carlsbad, Calif.) and Alexa Fluo 597 donkey anti-sheep IgG (1:200 in 5% dry milk, Invitrogen) for 4 hours. After washing, sections were placed in an autofluorescence-reduction solution (10 mM CuSO4, 50 mM ammonium acetate) for 30 minutes, and then mounted with Slow Fade Gold Antifade Reagent with DAPI (Invitrogen). The sections were viewed on Zeiss Axiovert 200 fluorescent microscope and MetaMorph imaging system (Universal Imaging Corporation, West Chester, Pa.). Negative controls included sections in which the primary antibodies were omitted (incubated with secondary antibodies only), or sections in which both primary and secondary antibodies were omitted. Further negative controls included use of sEH primary antibody after preincubation with its specific blocking peptide, and use of tissue from a sEHKO mouse heart. No sEH signal was detectable in any of the negative controls.

### Statistics

Data were analyzed using a commercially available statistics program (Prism 5.0). Differences between groups were assessed with Student's t-test and one-way ANOVA with post-hoc Bonferroni test as appropriate. All data were expressed as mean±SEM unless otherwise noted. A value of p<0.05 was considered to indicate statistical significance.

### Results

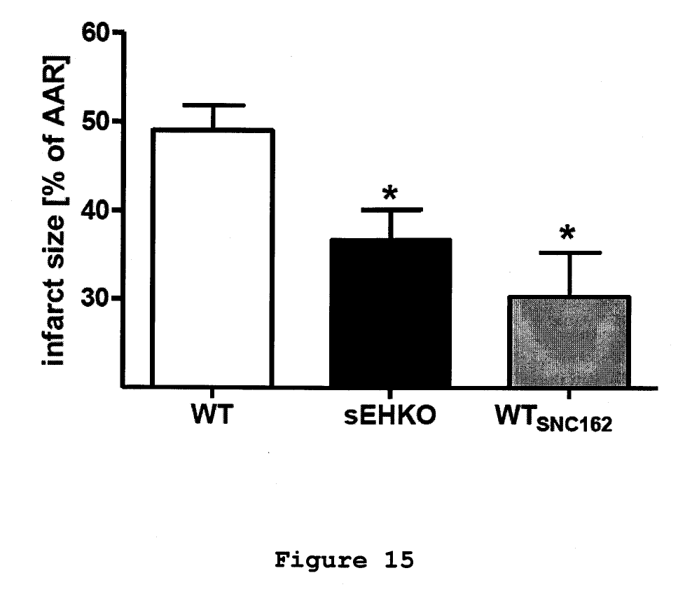

sEH gene deletion reduces infarct size after myocardial I/R. sEHKO mice sustained significantly reduced I/AAR compared to WT C57BL\6J mice. The observed reduction of I/AAR size in sEHKO mice was comparable to the effect of pre-ischemic administration of SNC-162 (WT_{SNC}) (FIG. 15). The ischemic insult was similar in both groups as evidenced by similar AARs normalized to biventricular heart volume (Table 3). Physiologic parameters recorded at baseline are shown in Table 4. Temperature was kept at 37° C. during the entire duration of the experiment and showed no difference between WT, sEHKO and WT_{SNC} mice.

TABLE 3
Weight and volume.

| | | Body | Biventricular | Area At | Infarct | |
|---|---|---|---|---|---|---|
| | | Weight | Weight | Risk | Volume | AAR/Biventricular |
| | | (g) | (mg) | (mm³) | (mm³) | volume (%) |
| | WT | 28 ± 1 | 114 ± 5 | 39 ± 5 | 18 ± 2 | 29 ± 3 |
| | sEHKO | 25 ± 1* | 115 ± 3 | 35 ± 4 | 13 ± 2 | 27 ± 3 |
| | WT_{SNC} | 26 ± 1 | 115 ± 6 | 31 ± 3 | 9 ± 2 | 27 ± 2 |
| PRE | vehicle | 28 ± 1 | 131 ± 3 | 38 ± 4 | 21 ± 3 | 28 ± 3 |
| | 14,15-EET | 27 ± 0 | 130 ± 4 | 36 ± 2 | 10 ± 1 | 29 ± 1 |
| PRO | vehicle | 28 ± 0 | 128 ± 2 | 43 ± 3 | 20 ± 2 | 32 ± 3 |
| | 14,15-EET | 27 ± 1 | 130 ± 5 | 33 ± 6 | 12 ± 2 | 26 ± 4 |
| PRE | vehicle | 27 ± 1 | 117 ± 3 | 43 ± 2 | 20 ± 2 | 33 ± 1 |
| | AUDA-BE | 27 ± 1 | 123 ± 3 | 39 ± 3 | 12 ± 2 | 28 ± 2 |
| PRO | vehicle | 27 ± 0 | 121 ± 3 | 40 ± 2 | 19 ± 1 | 30 ± 2 |
| | AUDA-BE | 26 ± 1 | 121 ± 9 | 45 ± 3 | 15 ± 3 | 34 ± 1 |

Shown are the body weight, wet biventricular weight, area-at-risk (AAR), infarct volume and AAR as percentage of biventricular volume for all 5 experiments (mean ± SEM;
*p < 0.05 compared to WT).

TABLE 4
Heart rate and rectal temperature at baseline.

| | | HR | T |
|---|---|---|---|
| | sEHKO | 535 ± 16* | 37.1 ± 0.1 |
| | WT | 619 ± 12 | 37.0 ± 0.1 |
| | WT_{SNC} | 494 ± 18* | 36.9 ± 0 |
| PRE | vehicle | 581 ± 12 | 37.1 ± 0 |
| | 14,15-EET | 600 ± 10 | 37.1 ± 0.1 |
| POST | vehicle | 514 ± 33 | 36.9 ± 0.1 |
| | 14,15-EET | 541 ± 27 | 36.9 ± 0.1 |
| PRE | vehicle | 496 ± 17 | 37.0 ± 0 |
| | AUDA-BE | 555 ± 24* | 37.0 ± 0.1 |
| POST | Vehicle | 543 ± 12 | 36.8 ± 0 |
| | AUDA-BE | 490 ± 27 | 36.9 ± 0.1 |

Shown are the heart rate (HR) in beats per minutes (BMP) and the rectal temperature (T) in degree Celsius (° C.) for all 5 experimental groups (mean ± SEM). No differences in corresponding groups were found.

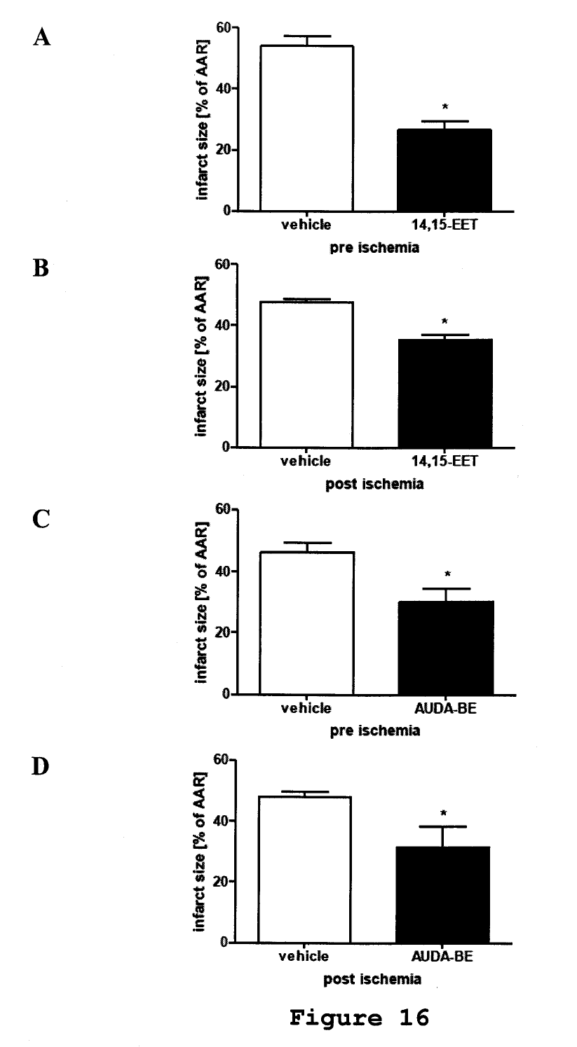

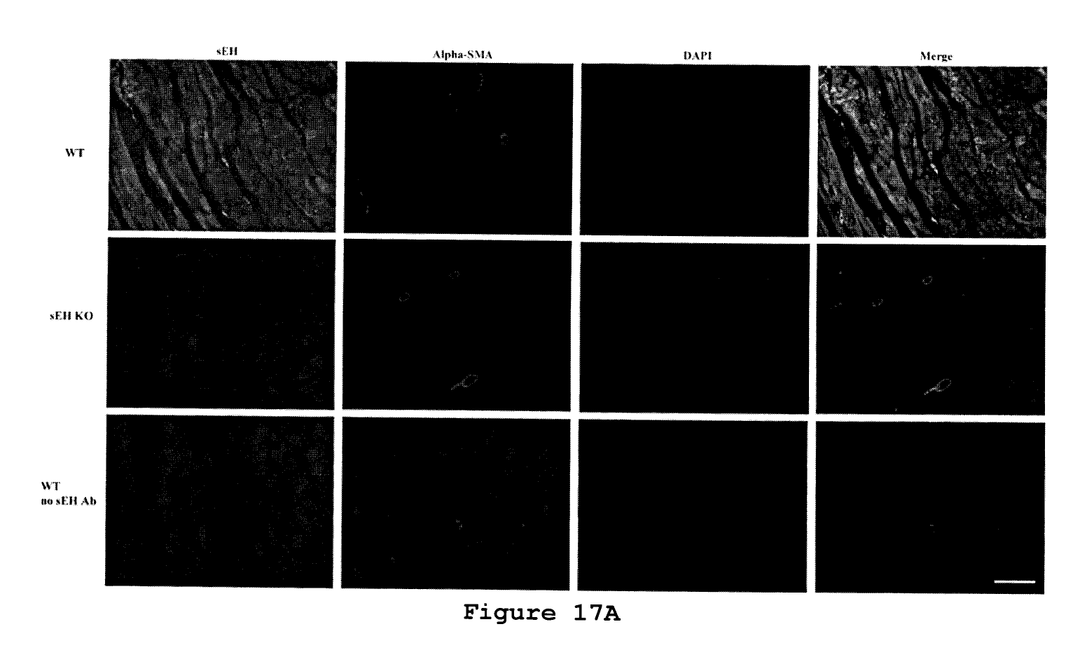

14,15-EET reduces myocardial infarct size independent of timing of treatment. Infarct size was significantly reduced by 14,15-EET compared to vehicle whether 14,15-EET was administered prior to ischemia or prior to reperfusion, as shown in FIG. 16. However, 14,15-EET injection prior to reperfusion was less effective in reducing I/AAR as compared to pre-ischemic delivery. The ischemic insult was similar in all 4 groups tested, as shown by similar AAR normalized to biventricular heart volume (Table 3).

Inhibition of sEH reduces myocardial infarct size independent of timing of treatment. Administration of AUDA-BE significantly reduced infarct size after myocardial I/R compared to vehicle control whether it was given before ischemia or before reperfusion (FIG. 16). Infarct size did not significantly differ between pre-ischemic and pre-reperfusion administration of AUDA-BE. The ischemic insult was similar in all 4 groups tested, as shown by similar AAR normalized to biventricular heart volume (Table 3).

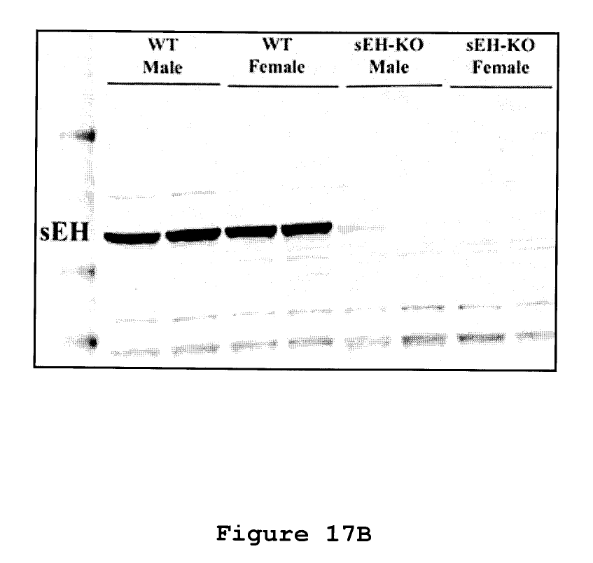

Expression and regional distribution of sEH in left ventricular tissue. Immunohistochemistry demonstrated that sEH is abundantly expressed in WT left ventricular cardiomyocytes, but absent in sEHKO hearts (FIG. 17A). Co-immunostaining with α-SMA antibody, which stains myofibroplasts showed no enhancement for sEH in this cell type. DAPI was used to visualize the nucleus, which did not enhance for sEH. The presence of sEH in left ventricular tissue was also confirmed by immunoblotting, which showed robust expression of sEH protein in WT hearts and the absence of sEH protein in sEHKO hearts (FIG. 17B).

These findings indicate: 1) sEH gene deletion is protective against regional myocardial ischemia-reperfusion injury in vivo; 2) pharmacological inhibition of sEH is cardioprotective whether the inhibitor is administered prior to or at the end of ischemia; 3) exogenously administered 14,15-EET reduces infarct size in a collateral-deficient model of regional myocardial ischemia-reperfusion, when administered prior to or at the end of LCA occlusion; and 4) sEH is abundantly expressed in left ventricular cardiac myocytes from WT. These findings also indicate that sEH and its main substrate 14,15-EET are important modulators of myocardial ischemia-reperfusion injury, and that strategies aimed at increasing 14,15-EET, including sEH inhibition, can serve as a therapeutic strategy in myocardial I/R injury.

The instant invention provides clear indications that augmentation of the levels of P450 epoxygenase products EETs, either through inhibition or gene deletion of sEH or through administration of exogenous EETs, promotes tolerance to ischemic stress. For instance, intraperitoneal injection of AUDA-BE (10 μg/g, the dose used in the current study) effectively reduces sEH activity in brain tissue and shows neuroprotective effects in an experimental stroke model in vivo, as described hereinabove. Similarly, sEHKO mice sustain reduced infarct size after experimental stroke in vivo, as described herein. Augmentation of endogenously derived EETs by CYP2J2 epoxygenase overexpression improves functional recovery in isolated heart preparations after global ischemia-reperfusion (Seubert et al. (2004) Circ. Res., 95:506-514). Furthermore, it has been shown that pre-ischemic administration of exogenous 14,15-EET reduces infarct size in vivo in rat and dog models of regional myocardial ischemia-reperfusion (Nithipatikom et al. (2006) Am. J. Physiol. Heart Circ. Physiol., 291:H537-H542; Gross et al. (2007) J. Mol. Cell Cardiol., 42:687-691). Notably, these other studies demonstrate infarct reduction by EETs administration when EETs were given pre-occlusion in rats (Gross et al. (2007) J. Mol. Cell Cardiol., 42:687-691), and both pre-occlusion and pre-reperfusion in dogs (Nithipatikom et al. (2006) Am. J. Physiol. Heart Circ. Physiol., 291:H537-H542). Further, dogs possess a variable and frequently robust coronary collateral circulation. The instant data demonstrate the “post-ischemic” protective effect of EETs in a collateral-deficient mouse model of regional I/R. The current data also show that administration of EETs at the onset of reperfusion imparts tolerance to myocardial ischemia-reperfusion in a collateral deficient species, suggesting that the salutary effect is indeed a direct cardiomyocyte phenomenon.

Soluble epoxide hydrolase is a bifunctional enzyme with a hydrolase activity located in the C-terminal and a phosphatase activity in the N-terminal (Newman et al. (2003) Proc. Natl. Acad. Sci., 100:1558-1563). Most of the known biological functions of sEH are attributed to its hydrolase activity. The main substrate for sEH is 14,15-EET, resulting in rapid hydration to 14,15-dihydroxyeicosatrienoic acid (14,15-DHET). Cardioprotection mediated by sEH inhibition is likely mainly due to augmenting endogenous 14,15-EET. For example, the 14,15-EET antagonist 14,15 epoxyeicosa-5(Z)-enoic acid (14,15-EEZE) abolished the improved recovery of left ventricular developed pressure in isolated hearts from sEHKO mice (Seubert et al. (2006) Circ. Res., 99:442-450). The cardioprotective effect of EETs is linked to activation of multiple cell survival pathways, including activation of mitochondrial and sarcolemmal KATP channels, calcium-sensitive potassium channels and phosphatidylinositol-3 kinase (Seubert et al. (2006) Circ. Res., 99:442-450). In addition, EETs have a wide range of other properties which may in part mediate the observed cardioprotection, including reduced generation of reactive oxygen species (Spiecker et al. (2005) Arch. Biochem. Biophys., 433:413-420), anti-inflammatory (Node et al. (1999) Science 285:1276-1279), antipyretic (Kozak et al. (2000) Am. J. Physiol. Regul. Integr. Comp. Physiol., 279:R455-R460), anti-platelet (Krotz et al. (2004) Arterioscler. Thromb. Vasc. Biol., 24:595-600), and anti-apoptotic functions (Chen et al. (2001) Mol. Cell Biol., 21:6322-6331).

Soluble epoxide hydrolase is known to be expressed in several tissues including blood vessels (Enayetallah et al. (2006) J. Histochem. Cytochem., 54:329-335). In the brain it has been localized in neurons (Koerner et al. (2007) J. Neurosci., 27:4642-4649). sEH activity has been detected in heart tissue by immunoblot (Seubert et al. (2006) Circ. Res., 99:442-450). However, it is not clear which cell type constitutes the source for sEH. Herein, it has been determined that sEH is abundantly expressed in left ventricular tissue from WT mice and is absent in hearts from sEHKO mice. Further, it is demonstrated herein that Seh localizes in cardiomyocytes from left ventricular tissue by immunohistochemistry. This observation is consistent with the ability of sEH inhibition to induce cardioprotection independent of vasodilatation. sEH expression was not observed in coronary vessels, but the main enhancement was found in cardiomyocytes.

The primary endpoint in the present study was reduction in infarct size. Because EETs are vasodilators, it is possible that sEH inhibition or gene deletion may have resulted in a decrease in post-ischemic blood pressure. However, if hemodynamically significant, this would have resulted in reduced coronary perfusion pressure and increased infarct size, which was not the case. Although sEH inhibition is expected to augment endogenous EETs, the effect will be most pronounced in tissues with high EETs production, such as ischemic tissue. Thus, regional myocardial ischemia should limit the augmentation of EETs primarily to the ischemic area within the heart. Therefore, although EETs are vasodilatory, systemic vasodilation and hypotension are not expected. Indeed, it has been reported that there is no differences in heart rate and blood pressure at 30 minutes of occlusion and 2 hours of reperfusion in a rat model of regional myocardial I/R in between groups tested with a similar dose of 14,15-EET (Gross et al. (2007) J. Mol. Cell Cardiol., 42:687-691). In contrast to regional myocardial ischemia, where sEHKO mice are protected, in cardiac arrest-induced whole-body ischemia, sEHKO mice were at a disadvantage, presumably due to excessive vasodilation and an inability to maintain blood pressure after cardiopulmonary resuscitation.

AUDA-BE is a newly developed potent and selective sEH inhibitor (Kim et al. (2004) J. Med. Chem., 47:2110-2122). The pharmacokinetic profile in C57BL\6J mice has recently been characterized and the dose used in the current study (10 μg/g) results in plasma levels greater than the IC₅₀ for the inhibitor within 1 hour of administration. The fact that both sEH inhibition and exogenously administered 14,15-EET resulted in reduced infarct size when given during ischemia prior to reperfusion suggests that this approach is useful clinically, for example prior to percutaneous coronary interventions or surgical revascularization in the setting of acute myocardial ischemia.

In summary, the findings in the present study represent the first description of successful cardioprotection by sEH inhibition before and during ischemia, and the first demonstration of in vivo cardioprotection by sEH gene deletion. The data support the concept of using sEH inhibition as a novel therapeutic treatment for myocardial cardioprotection.

### EXAMPLE 6

### Materials and Methods

The study was conducted in accordance with the National Institutes of Health guidelines for the care and use of animals in research, and protocols were approved by animal care and use committee of Oregon Health and Science University, Portland, Oreg.

### Animals

Mice with targeted deletion of soluble epoxide hydrolase (sEH knockout, sEHKO) have been described (Sinal et al. (2000) J. Biol. Chem., 275:40504-40510). The mice originated on a B6; 129X1 background and have been backcrossed to C57BL/6 for at least 6 generations, and therefore, homozygous sEHKO mice were compared to WT C57BL/6 mice obtained from The Jackson Laboratories. Homozygous mice are viable, fertile, normal in size and do not display any gross physical or behavioral abnormalities. Mice were genotyped by PCR as previously described (Sinal et al. (2000) J. Biol. Chem., 275:40504-40510).

### MCAO in Mice

Transient focal cerebral ischemia was induced in adult male mice (20 to 26 g body weight) using the intraluminal MCAO technique, as previously described (Alkayed et al. (2001) J. Neurosci., 21:7543-7550). Briefly, mice were anesthetized with halothane (1.5 to 2% in O₂-enriched air by face mask), and kept warm with water pads. A small laser-Doppler probe was affixed to skull to monitor cortical perfusion and verify vascular occlusion and reperfusion. A silicone-coated 6-0 nylon monofilament was inserted into the right internal carotid artery (ICA) via the external carotid artery (ECA) until laser-Doppler signal dropped to <30% of baseline. After securing the filament in place, the surgical site was closed, and the animal was awakened and assessed at 2 hours of occlusion for neurological deficit using a simple neurological scoring system as follows: 0=no deficit, 1=failure to extend forelimb, 2=circling, 3=unilateral weakness, 4=no spontaneous motor activity. Mice with neurological deficit score between 1 to 3 were reanesthetized, laser-Doppler probe repositioned over same site on the skull, and the occluding filament was withdrawn to allow for reperfusion. Mice were then allowed to recover and were observed for 1 day. Infarct size was measured at 24 hours after MCAO in 2-mm thick coronal brain sections (5 total) using 2,3,5-triphenyltetrazolium chloride (TTC) staining and digital image analysis. Sections were incubated in 1.2% TTC in saline for 15 minutes at 37° C., and then fixed in formalin for 24 hours. Slices were photographed and infracted (unstained) and uninfarcted (red color) areas were measured with MCID software (InterFocus, Linton, England) and integrated across all 5 slices. To account for the effect of edema, infarct size was estimated indirectly by subtracting the uninjured area in the ipsilateral hemisphere from the contralateral hemisphere, and expressing infarct volume as a percentage of the contralateral hemisphere. Separate groups of nonsurvival WT and sEHKO mice (n=5 per group) were kept under anesthesia for 2 hours to continuously monitor laser-Doppler cerebrocortical tissue perfusion and arterial blood pressure and gases. To determine whether 14,15-EET is protective against ischemic brain injury, synthetic 14,15-EET (1 μg) was administered over 24 hours via jugular vein catheter connected to a subcutaneously implanted osmotic minipump. To determine whether protection in sEHKO mice was mediated via 14,15-EET, the effect of 14,15-EET antagonist 14,15 epoxyeicosa-5(Z)-enoic acid 14,15-EEZE (10 μg over 24 hours via minipump implanted 1 hour before MCAO; Gauthier et al. (2003) Hypertension 42:555-561) was examined.

### Western Blot and Immunohistochemistry

Brain tissue from WT versus sEHKO mice were homogenized in solution A containing sucrose (250 mmol/L), KCl (60 mmol/L), Tris HCl (15 mmol/L), NaCl (15 mmol/L), EDTA (5 mmol/L), EGTA (1 mmol/L), PMSF (0.5 mmol/L), and DTT 10 mmol/L), then centrifuged at 2000 g for 10 minutes at 4° C. Protein samples (30 μg) were separated by SDS-polyacrylamide gel electrophoresis and transferred to PVDF membranes. Blots were then blocked in 5% dry milk and incubated at 4° C. overnight with a primary rabbit anti-sEH antibody (Draper et al. (1999) Toxicol. Sci., 50:30-35; Pinot et al. (1995) Biochem. Pharmacol., 50:501-508). Signal was visualized using a biotinylated secondary antibody (Amersham) with an ECL plus chemiluminescence detection kit (Amersham). Autoradiograms were scanned and band optical densities quantified with Quantityone software (BioRad). Blots were reprobed for β-actin (Sigma) to ensure equal loading. To localize sEH immunoreactivity in brain, thin coronal sections were probed with anti-sEH antibody.

### Atmospheric Pressure Chemical Ionization (APCI) Liquid Chromatography-Tandem Mass Spectrometry (LC-MS/MS)

The amount of free 14,15-EET from brain tissue of WT versus sEHKO mice was determined using electron capture APCI LCMS/MS of the pentafluorobenzyl-esters (PFB) of the EETs (Lee et al. (2003) Rapid Commun. Mass Spectrom., 17:2168-2176.). The extraction method of Yue et al. was adopted (Yue et al. (2004) J. Chromatogr. B Analyt. Technol. Biomed. Life Sci., 803:267-277). Briefly, frozen brain tissue (approximately 50 mg) was added to 200 μL methanol containing 0.2% formic acid and 1000 pg (±) 14,15-EET-d8 (BioMol Research Laboratories) internal standard was added. The tissue was homogenized on ice with a microultrasonic cell disruptor at a setting of 6.5 (2 mm probe, highest setting 20, Microscan Ultrasonic). Following centrifugation at 14,000 rpm for 15 minutes, the supernatants were diluted with water to a final methanol concentration of 10% and loaded onto a 1 mL Oasis HLB SPE cartridge reconditioned as described (Yue et al. (2004) J. Chromatogr. B Analyt. Technol. Biomed. Life Sci., 803:267-277). The cartridges were washed with 1 mL of water and 10% methanol, and dried under nitrogen for 30 minutes. The metabolites were eluted with anhydrous acetonitrile and dried under nitrogen. Dried extracts were dissolved in 100 μL of anhydrous acetonitrile and derivatized at 60° C. after the addition of 100 μL of 2.5% 2,3,4,5,6-pentafluorobenzyl (PFB) bromide (99% Aldrich) in anhydrous acetonitrile and 5% N,N-diisopropylethylamine (redistilled, 99.5% Aldrich) in anhydrous acetonitrile (Lee et al. (2003) Rapid Commun. Mass Spectrom., 17:2168-2176.). The reaction was cooled to room temperature and the PFB-esters were extracted with hexane after the addition of equal volume of 0.15 mol/L KCl solution. The hexane extract was dried and the residue dissolved in 50 μL of 85% methanol for analysis by LC-MS/MS. EET standards were obtained from Cayman. The PFB-esters of DHETS, HETEs and EETs were resolved using a Surveyor HPLC system with a 50×2.1 mm (5 μm) Betabasic C18 HPLC column (ThermoHypersil) at a flow rate of 0.3 mL/min with a gradient from 75% to 98% methanol over 35 minutes. Mass spectrometry was conducted with a TSQ Quantum Discovery triple-quadrupole mass spectrometer equipped with an APCI source in negative mode. The instrument operating conditions were as follows: vaporizer temperature, 350° C.; capillary temperature, 200° C.; capillary offset, −35 V; corona discharge needle, set at 18 μA. The sheath and auxillary gas were 35 psi and 2 arbitrary units, respectively. Collision induced dissociation was performed with argon as the collision gas at 0.8 mTorr in the second quadropole. Unit resolution was maintained for precursor and products ions. 14,15-EET was monitored in the selected reaction monitoring mode with transitions of m/z=319-175 as well as m/z=319-219. The deuterated internal standard, 14,15-EET-d₈, was monitored with the transition of m/z=327-226. The amount of 14,15-EET was calculated by comparison of the area ratio of 14,15-EET to 14,15-EET-d₈ to those obtained when brain tissue was spiked with known amounts of 14,15-EET.

### 14,15-DHET ELISA

Total 14,15-DHET in blood was measured with a commercial ELISA (Detroit R&D, Inc, Detroit, Mich.) (Koerner et al. (2007) J. Neurosci., 27:4642-4649). Briefly, plasma lipids were extracted 3 times with ethyl acetate under acidic conditions (pH 3 to 4), followed by saponification with 20% KOH. Samples were dried and resuspended in methanol, and 14,15-DHET concentration was measured according to the manufacturer's instructions. The ELISA was also used to measure hydrolase activity in brain tissue (perfused to remove blood) incubated with 1 μM 14,15-EET for 30 minutes. Brian tissue was homogenized in 4 volumes of ice-cold buffer (20 mmol/L Tris HCL pH 7.4, 0.32 mol/L sucrose, 1 mmol/L EDTA), centrifuged at 1000 g for 10 minutes, and supernatant was further centrifuged at 10,000 g for 20 minutes.

### Regional CBF Measurement Using [¹⁴C]-Iodoantipyrine (IAP) Autoradiography

End-ischemic regional CBF was measured in a nonsurvival cohort of sEHKO or C57Blk/6 mice using quantitative autoradiography with [¹⁴C] IAP, as described (Zhang et al. (2007) J. Cereb. Blood Flow. Metab., 27:1931-1949). Mice were instrumented with femoral artery and jugular vein catheters, and MCA occluded as in the previous cohort. At 2 hours of MCA occlusion, 1 μCi of [¹⁴C] IAP in 75 μL of saline was infused intravenously for 45 seconds. Free-flowing arterial blood was simultaneously sampled at 5-second intervals for the arterial input function. With the filament in place, mice were decapitated at 45 seconds after the start of infusion, and the brain was quickly removed and frozen in 2-methylbutane on dry ice. Brains were sectioned on a cryostat into 20-μm-thick coronal slices, which were thaw-mounted on cover glass and then apposed for 3 weeks to film (Kodak, Bio-Max) together with ¹⁴C standards. The concentration of [¹⁴C] IAP in blood was determined by liquid scintillation spectroscopy (model 6500, Beckman) after decolorization with 0.2 mL of tissue solubilizer (Soluene-350, PerkinElmer, Inc). Autoradiographic images representing 5 different coronal levels (+2, +1, 0, −1 and −2 from Bregma, 3 images each) were digitized, and rCBF was determined in specific regions with the use of image analysis software (MCID, 7.0). Additionally, areas with similar flow rates were isolated and summed to construct a histogram of brain tissue volumes perfused with given blood flow rates.

### Statistical Analysis

Differences in infarct size, DHET levels and CBF rates were analyzed with at test for 2 groups and analysis of variance (ANOVA) with post hoc Newman-Keuls multiple range test for multiple groups. The criterion for statistical significance is P<0.05. All values are reported as mean±SEM.

### Results

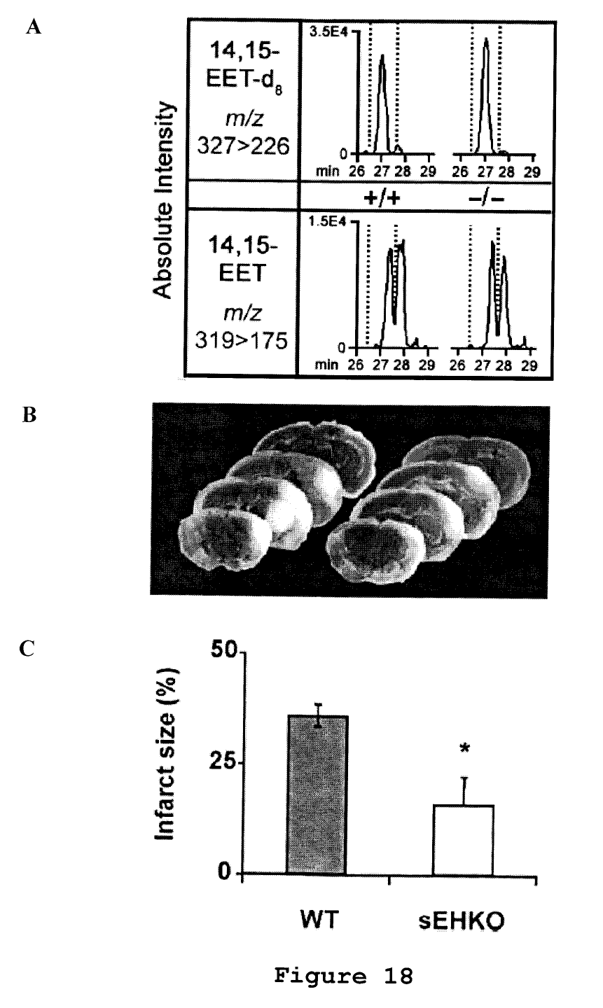

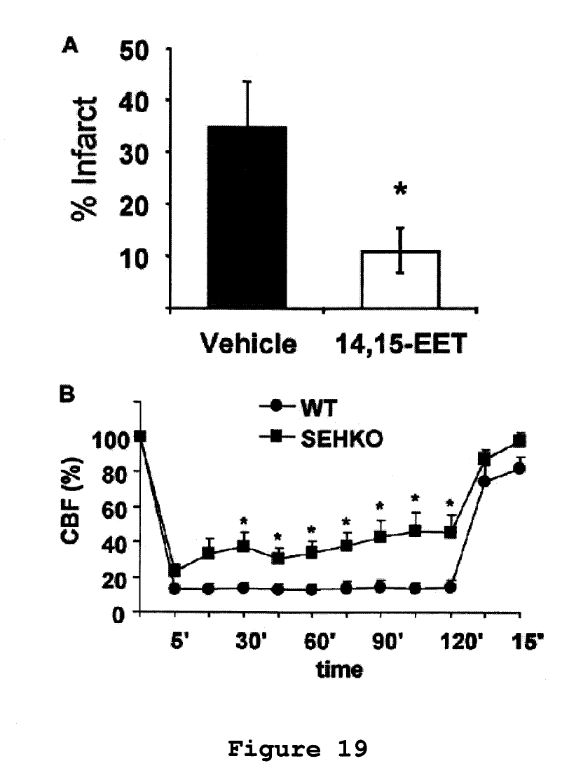

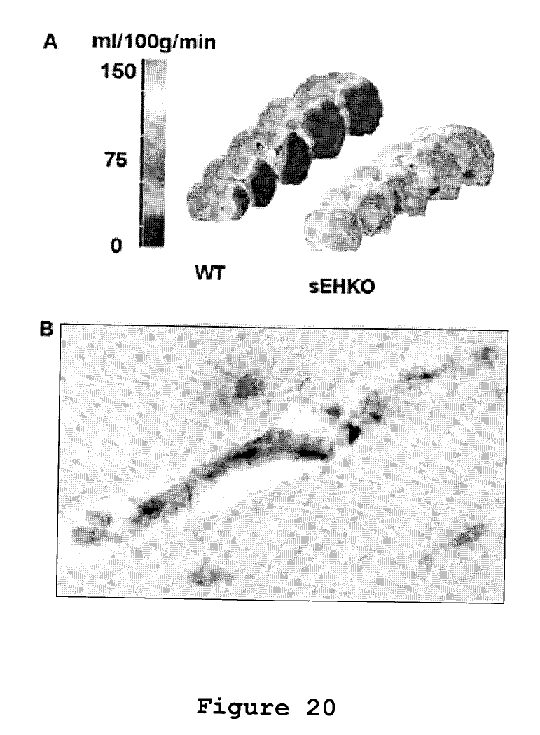

PCR-based genotyping strategy of WT (+/+, 233 bp) and sEHKO mice (−/−, 190 bp) and Western blotting demonstrates that sEH protein is abundantly expressed in WT, but not in homozygous sEHKO mouse brain (62.5 kDa, n=4). FIG. 18A shows representative LC-MS/MS extracted ion chromatograms (XICs) depicting free endogenous brain 14,15-EET peaks in WT and SEHKO mouse brains. The levels of free 14,15-EET detected in brain tissue were not statistically different between WT and sEHKO mice: 483 pg per 50 mg tissue (±SD 93 pg, n=3) compared to 735 pg (SD 382 pg, n=3), respectively. Similarly, there were no differences in brain tissue levels of other EETs regioisomers between WT and sEHKO mice. Furthermore, there were no differences in brain hydrolase activities between WT and sEHKO mice, as indicated by the ability of brain tissue from these mice to convert 14,15-EET to corresponding DHET ex vivo. Interestingly, the metabolism of 14,15-EET to 14,15-DHET in both WT and KO brain tissue was inhibited by the epoxide hydrolase inhibitor AUDA-BE (Zhang et al. (2007) J. Cereb. Blood Flow. Metab., 27:1931-1949). Finally, the levels of total 14,15-DHET in WT and sEHKO mouse plasma was measured using ELISA (Koerner et al. (2007) J. Neurosci., 27:4642-4649). Total 14,15-DHET in plasma was significantly lower in sEHKO than WT mice (115±73, n=10, versus 4179±482 pg/mL, n=12, P<0.001). FIGS. 18B and 18C demonstrates that sEHKO mice sustained a smaller infarct after MCAO compared to WT mice (16±6% compared to 36±3%, respectively, n=5 per group, P<0.05). Neurological deficit during and at 24 hours after MCAO was significantly smaller in sEHKO compared to WT mice. FIG. 19A illustrates that exogenous 14,15-EET significantly reduces infarct size after MCAO in mice from 34.5±8.6% to 10.9±4.3% (n=4 per group, P<0.05). However, 14,15-EEZE administration did not alter infarct size (9.4±12.4 [mean±SD, n=9]) compared to vehicle-treated sEHKO mice (9.1±9.5, n=5, P<0.97). Laser-Doppler monitoring in surviving WT and sEHKO mice suggested that tissue perfusion was reduced to the same degree in both groups. However, continuous monitoring of laser-Doppler perfusion for the entire 2-hour MCAO period revealed that laser-Doppler perfusion remained stably low in WT mice, whereas tissue perfusion slowly increased in sEHKO mice, and by 30 minutes was significantly higher than WT mice (FIG. 19B). To confirm that blood flow rates were higher in sEHKO versus WT mice at the end of MCAO, [¹⁴C]iodoantipyrine (IAP) autoradiography was used to determine absolute regional CBF rates in WT and sEHKO mouse brain at the end of 2-hour MCAO. FIG. 20A is distribution of CBF in ischemic and contralateral hemispheres, demonstrating higher tissue perfusion in ipsilateral hemisphere at end ischemia in sEHKO compared to WT mice (representative of 5 mice per group). Analysis of blood flow distribution in the two strains revealed that the amount of tissue perfused with ischemic flow rates (0 to 20 mL/100 g/min) were significantly lower in sEHKO compared to WT mice at the end of MCAO (P<0.05, n=5). Finally, immunohistochemical analysis localized sEH expression in cerebral blood vessels (FIG. 20B).

Therefore, the above demonstrates, among other things: (1) targeted deletion of sEH is protective against focal cerebral ischemia, although protection was not diminished by EETs antagonist 14,15-EEZE, (2) brain tissue perfusion is higher in sEH null compared to WT mice during vascular occlusion, (3) brain tissue hydrolase activity and levels of free 14,15-EET were not different between sEHKO and WT mice, despite the absence of sEH immunoreactivity in knockout mouse brain, (4) the level of total 14,15-DHET in blood was lower in sEHKO compared to WT mice, and (5) immunoreactivity was detected in cerebral vessels from WT mice. Accordingly, it can be concluded that sEH gene deletion is protective against ischemic brain injury, in part due to reduced metabolism of circulating 14,15-EET and enhanced vasodilator capacity and collateral blood flow in response to focal vascular occlusion. The findings also indicate that circulating 14,15-EET serves a protective function, and that manipulations aimed at decreasing its metabolism is protective against ischemic stroke.

The lack of difference in 14,15-EET between WT and sEHKO mice suggests that sEHKO mouse brain may have an alternative mechanism for EETs hydrolysis other than sEH. This enzymatic activity, however, was also inhibited by sEH inhibitor AUDA-BE, and was present in sEHKO brain but not peripheral tissue. The source of circulating EETs and tissue localization of hydrolase activity responsible for DHET in blood is unclear. The most obvious source for EETs is vascular endothelium and the most obvious location of sEH is vascular smooth muscle, although contributions from liver, kidney, lungs and other tissues, including blood cells, cannot be ruled out. In support of a vascular site for EETs hydrolysis, immunohistochemistry localized sEH in cerebral blood vessels. This is consistent with the study demonstrating sEH expression in both vascular and nonvascular brain compartments (Zhang et al. (2007) J. Cereb. Blood Flow Metab., 27:1931-1949). The identity of the residual hydrolase activity in sEHKO brain is unknown. One possibility is that the microsomal epoxide hydrolase (mEH), which can potentially metabolize 14,15-EET, albeit less efficiently than sEH, may assume a more prominent role in EETs metabolism in brain when sEH is deleted. The fact that no such compensation takes place in peripheral tissues may be related to the differential levels of expression or regulation of mEH in neural versus peripheral tissues.

To determine whether 14,15-EET is responsible for the protection in sEHKO mice, the 14,15-EET antagonist 14,15-EEZE was used. However, 14,15-EEZE administration did not increase infarct size in sEHKO mice. This may indicate that protection in sEHKO mice is not mediated through 14,15-EET, or it may be related to the observation that 14,15-EEZE exhibits agonistic activity in some tissues (Harrington et al. (2004) Eur. J. Pharmacol., 506:165-168). In the above study, 14,15-EET levels in blood was not measured using LC-MS/MS. However, a recent report has demonstrated using LC-MS/MS that the levels of epoxy fatty acids, including 14,15-EET, were elevated in blood, liver and kidney in two sEHKO mouse lines, including the one used in this study (the “NIH” line), compared to WT mice (Luria et al. (2007) J. Biol. Chem., 282:2891-2898). It should be noted, however, that despite the evidence that protection in sEHKO mice was associated with reduced circulating 14,15-DHET, and that it was recapitulated by exogenous 14,15-EET administration, the data does not completely exclude contributions by other EETs regioisomers. It is also possible that the protection may not at all be related to EETs, since sEH is a bifunctional enzyme, with a C-terminal hydrolase and an N-terminal phosphatase. Nevertheless, data are still limited regarding possible endogenous substrates for the N-terminal phosphatase (Enayetallah et al. (2006) Biochem. Biophys. Res. Commun., 341:254-260), and attempts to elucidate the enzyme's phosphatase function and relevance to stroke are hampered by the inability of known phosphatase inhibitors to block the N-terminal phosphatase activity of sEH (Tran et al. (2005) Biochemistry 44:12179-12187.).

The finding here that sEH gene deletion is protective against ischemic brain injury is consistent with the finding that pharmacological inhibition of sEH reduces infarct size after MCAO in WT mice, and that polymorphisms in the sEH gene that alter the enzyme's hydrolase activity are linked to neuronal survival after ischemic injury. This is also consistent with the demonstration of improved functional recovery after myocardial ischemia in isolated heart preparation from sEHKO mice. The finding that 14,15-EET is protective in vivo is also consistent with the observation that 14,15-EET is protective against cell death induced in primary cortical neurons and astrocytes by oxygen-glucose deprivation (OGD). However, the mechanism of neuroprotection by 14,15-EET in vitro is likely different than its mechanism of action in vivo. The data using laser-Doppler perfusion and iodoantipyrine autoradiography suggest that the mechanism of protection in vivo may be related to EETs' vasodilator property. This, however, is in contrast to the previous observation that pharmacological inhibition of sEH was protective against ischemic stroke by a blood flow-independent mechanism. The discrepancy may be related to the fact that sEH is a bifunctional enzyme, and that while gene deletion eliminates the enzyme's lipid phosphatase and epoxide hydrolase functions, the inhibitors were designed to inhibit only the hydrolase function. Another possibility is that the level of 14,15-EET achieved by complete gene deletion, and required for vasodilation, is much higher than that achieved by partial pharmacological inhibition. It should be noted, however, that EETs possess other properties that can potentially explain the protective effect of sEH deletion. For example, EETs have been shown to suppress inflammation, hyperthermia, platelet aggregation, generation of reactive oxygen species, and apoptosis, all of which are known to exacerbate ischemic brain injury.

In summary, the current study provides the first characterization of CBF and ischemic brain injury in sEH null mice, and demonstrates that sEH gene deletion is associated with smaller infarct, higher circulating, but not brain 14,15 EET, and higher blood flow during focal vascular occlusion. The findings suggest that vascular sEH may play an important role in regulating EETs' circulating levels, and may serve as a therapeutic target for increased tissue perfusion and protection from ischemic brain injury.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for treating or preventing a condition characterized by cerebral hyperperfusion by administering at least one agent which inhibits the EETs signaling pathway.

2. The method of claim 1, wherein said condition characterized by cerebral hyperperfusion is selected from the group consisting of migraine, cluster headaches, and primary headaches.

3. The method of claim 2, wherein said condition is migraine.

4. The method of claim 1, wherein said agent which inhibits the EETs signaling pathway is selected from the group consisting of agents which inhibit EETs-synthesizing enzymes, agents which inhibit the liberation of EETs from the phospholipid pool, agents which increase the activity of EETs-metabolizing proteins, and agents which inhibit the action of neurogenic EETs upon the cerebral artery.

5. The method of claim 4, wherein said agent is 14,15-epoxyeicosa-5(Z)-enoic acid.

6. The method of claim 1, further comprising the administration of at least one other migraine therapeutic agent.

7. A method for treating or preventing a condition characterized by cerebral hypoperfusion by administering at least one agent which activates the EETs signaling pathway.

8. The method of claim 7, wherein said condition characterized by cerebral hypoperfusion is selected from the group consisting of stroke, vasospasm after subarachnoid hemorrhage, and traumatic brain injury.

9. The method of claim 7, wherein said agent which activates the EETs signaling pathway is selected from the group consisting of agents which increase the activity of EETs-synthesizing enzymes, agents which increase the liberation of EETs from the phospholipid pool, agents which inhibit EETs-metabolizing proteins, and agents which increase the action of neurogenic EETs upon the cerebral artery.

10. The method of claim 9, wherein said agent inhibits soluble epoxide hydrolase (sEH).

11. The method of claim 9, wherein said agent is 14,15-EET.

12. A method for treating or preventing a condition characterized by inappropriately dilated blood vessels by administering at least one agent which inhibits the EETs signaling pathway.

13. The method of claim 12, wherein said condition characterized by inappropriately dilated blood vessels is selected from the group consisting of vasodilatory shock, the post-cardiac arrest state, and hypotension.

14. The method of claim 12, wherein said agent which inhibits the EETs signaling pathway is selected from the group consisting of agents which inhibit EETs-synthesizing enzymes, agents which inhibit the liberation of EETs from the phospholipid pool, agents which increase the activity of EETs-metabolizing proteins, and agents which inhibit the action of neurogenic EETs upon the cerebral artery.

15. A method for treating or preventing ischemia-reperfusion injury by administering to a patient in need thereof at least one agent which activates the EETs signaling pathway.

16. The method of claim 15, wherein said ischemia-reperfusion injury is cause by surgery, transplantation, or coronary arterial occlusion.

17. The method of claim 15, wherein said agent which activates the EETs signaling pathway is selected from the group consisting of agents which increase the activity of EETs-synthesizing enzymes, agents which increase the liberation of EETs from the phospholipid pool, and agents which inhibit EETs-metabolizing proteins.

18. The method of claim 17, wherein said agent inhibits soluble epoxide hydrolase (sEH).

19. The method of claim 17, wherein said agent is 14,15-EET.
